(19) United States
(12) Patent Application Publication
(10) Pub. No.: US 2021/0145764 A1
(43) Pub. Date: May 20, 2021

(54) COMPOSITIONS OF CANNABIDIOL (CBD), AND/OR POLYPHENOLS, AND METHODS FOR THE PREVENTION AND/OR TREATMENT OF SKIN, MUSCLE, NERVE AND INFLAMMATORY DISORDERS, AND BIOLOGICAL FACTORS AND FUNCTIONS IN MAMMALS

(71) Applicant: BRIGHAM YOUNG UNIVERSITY, Provo, UT (US)

(72) Inventor: Edwin Douglas LEPHART, Montgomery, TX (US)

(73) Assignee: BRIGHAM YOUNG UNIVERSITY, Provo, UT (US)

(21) Appl. No.: 17/096,535

(22) Filed: Nov. 12, 2020

Related U.S. Application Data
(60) Provisional application No. 62/936,222, filed on Nov. 15, 2019.

(51) Int. Cl. A61K 31/05; A61K 38/39; A61K 31/375; A61K 31/355; A61K 33/30; A61K 36/87; A61K 31/455; A61K 31/728; A61K 31/122; A61K 31/352; A61K 8/34; A61K 8/67; A61K 8/27; A61K 8/9789; A61K 8/365; A61K 8/35; A61K 8/73; A61K 31/19; A61Q 19/08; A61Q 19/02; A61Q 19/00; A61Q 3/00; A61Q 7/00; A61P 17/06; A61P 17/10; A61K 9/06; A61P 29/00; A61K 9/00; A61P 39/06; A61P 35/00

(52) U.S. Cl. CPC A61K 31/05; A61P 35/00; A61K 31/375; A61K 31/355; A61K 33/30; A61K 36/87; A61K 31/455; A61K 31/728; A61K 31/122; A61K 31/352; A61K 8/347; A61K 8/676; A61K 8/678; A61K 8/27; A61K 8/9789; A61K 8/675; A61K 8/365; A61K 8/355; A61K 8/735; A61K 31/19; A61Q 19/08; A61Q 19/02; A61Q 19/004; A61Q 3/00; A61Q 7/00; A61P 17/06; A61P 17/10; A61K 9/06; A61P 29/00; A61K 9/0014; A61P 39/06; A61Q 19/007; A61K 38/39

(57) ABSTRACT

Described herein are compositions and methods for the prevention and/or treatment of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject. The composition includes one or more polyphenolic compound, its isomer or analog; and one or more other active ingredient selected from the group consisting of collagen, vitamin C, vitamin E, zinc, grape seed extract, niacinamide, hydroxy acids, hyaluronic acid, and coenzyme Q10. The composition may also or alternatively include cannabidiol (CBD).

### RELATED APPLICATIONS

The present patent document claims the benefit of the filing date under 35 U.S.C. § 119(e) of Provisional U.S. Patent Application Ser. No. 62/936,222, filed Nov. 15, 2019, which is hereby incorporated by reference.

### BACKGROUND

Described herein are compositions including a polyphenolic compound, its isomer or analog, and one or more other active ingredient(s) selected from the group consisting of collagen, vitamin C, vitamin E, zinc, grape seed extract, niacinamide, hydroxy acids, hyaluronic acid, and coenzyme Q10 for the prevention, treatment and/or amelioration of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject. Also, described herein are compositions including cannabidiol (CBD) and at least one of: one or more polyphenolic compound, its isomer or analog; and one or more other active ingredient selected from the group consisting of collagen, vitamin C, vitamin E, zinc, grape seed extract, niacinamide, hydroxy acids, hyaluronic acid, and coenzyme Q10. Furthermore, described herein are methods of using the compositions for the prevention, treatment and/or amelioration of the at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject.

Plants have evolved a plethora of compounds that acts as protectants but also have generated a complex stress adaptive system to maintain dissipative homeostasis (Nunn et al., 2020).

Of the at least 113 cannabinoids that have been isolated to date, two are undoubtedly the most well-known and the most well researched, CBD and tetrahydrocannabinol (THC). Both are naturally occurring compounds found in plants in the Cannabis genus. Known as phytocannabinoids, these compounds interact with CB1 and CB2 receptors found in the endocannabinoid system present in all mammalian species (Rong et al., 2017; Burstein, 2019).

Cannabidiol (CBD) is obtained from the Cannabis plant that has high pharmacological and nutritional values based upon the biological active compounds that can be extracted from it. For example, Cannabis is known to contain cannafavins A, B and C, 3-sitosterol, vitexin, isovitexin, apigenin, keaempferol, quercetin, luteolin, and orientin.

However, the isoflavonoids or polyphenols such as genistein, daidzein and equol are not found in Cannabis plants (Pollastro et al., 2018).

Equol is one example of an isoflavonoid (has two phenolic rings with hydroxyl groups on each ring that provide functional points for biological activity (Lephart, 2013b; Lephart, 2016)). Equol can also be classified as a phytoestrogen. While endogenous estrogenic hormones such as 17β-estradiol are steroids, with a cyclo-hexane-phenantrene parent chemical structure that is derived from cholesterol, equol is not a steroid (Lephart, 2016). However, equol and 17β-estradiol have similar chemical structures/confirmations and molecular weights (C₁₅H₁₄O₃ vs. C₁₈H₂₄O₂; 242.3 g/mol vs. 272.4 g/mol, respectively) (Lephart, 2016; see FIG. 8).

Equol has a chiral center at carbon 3, and thus can exist in two mirror image forms known as enantiomers (S-equol and R-equol) (Lephart, 2013b; Setchell and Clerici, 2010, see FIG. 8). Equol can be produced as racemic equol which presumably, extract equal amounts of S-equol and R-equol (Lephart, 2013b; Lephart 2016). However, in man and animals only S-equol is produced by intestinal bacteria conversion from daidzein, and some individuals have the ability of producing higher levels of equol than others (Lephart, 2013b, Setchell and Clerici, 2010; Setchell et al., 2005). These individuals have been identified as “equol producer” (Lephart, 2013b, Setchell and Clerici, 2010). The term “equol producer” is a descriptive or arbitrary term for humans that maintain S-equol levels around or above 10 to 20 ng/ml after consumption of soy food products that infers protective health benefits (Lephart, 2013b; Lephart, 2016). S-Equol can be found in plant products such as beans, cabbage, lettuces, tofu and other food and animal products (Abiru et al., 2012; Common and Ainsworth, 1961; Hounsome et al., 2009; Hounsome et al., 2010; Jou et al., 2013).

For example, S-equol has been reported in cow's milk (Bannwart et al., 1986; HoikkE et al., 2007; Lundh et al., 1990; King et al., 1998), and Frankenfeld (2011) showed that dairy consumption significantly correlated with urinary equol levels in U.S. adults. Also, it has been shown that R-equol levels in fermented soy products are higher compared to S-equol (Lephart, 2013b, Lephart et al., 2014). Notably, the metabolism of R- and S-equol in humans appears to be similar (Setchell et al., 2009). Therefore, humans are exposed to this polyphenolic/isoflavonoid compound from different plant and food sources regardless of age, gender or geographical location with scientific data to support a consumption/exposure record that appears to be safe (Andres et al., 2012; Badger et al., 2009; Degen et al., 2011; Gilchrist et al., 2010; Hamilton-Reeves, 2010).

Both equol isomers and racemic equol have been shown to exhibit antioxidant, anti-inflammatory, skin protectant (against ROS/oxidative stress) and specifically anti-androgen hormonal actions by binding free 5α-dihydrotestosterone (5α-DHT) as a selective androgen modulator (SAM) (Gopaul et al., 2012; Lephart, 2013a; Lephart, 2016; Lephart, 2017; Lund et al., 2004; Lund et al., 2011; Magnet et al., 2017; Oyama et al., 2012). Notably, 5α-DHT that is unopposed is known to be toxic to mammalian cells such as fibroblasts (Gopaul et al., 2012).

### SUMMARY OF THE INVENTION

One embodiment relates to a composition comprising: (a) cannabidiol (CBD); and (b) at least one of: (i) one or more polyphenolic compound, its isomer or analog; and (ii) one or more other active ingredient selected from the group consisting of collagen, vitamin C, vitamin E, zinc, grape seed extract, niacinamide, hydroxy acids, hyaluronic acid, and coenzyme Q10.

Another embodiment relates to a composition comprising: (a) at least one polyphenolic compound, its isomer or analog; and (b) at least one other active ingredient selected from the group consisting of collagen, vitamin C, vitamin E, zinc, grape seed extract, niacinamide, hydroxy acids, hyaluronic acid, and coenzyme Q10. The composition may be in a form of lotion, spray, foam, gel, or trans-dermal patch, and is administered topically. The composition may be in the form of a liquid, tablet, capsule, dietary or nutritional supplement, and is administered orally. The composition may be administered by an intra-muscular injection or intra-venous methods. The composition may be administered as a food, supplement or OTC product, medicinal food, or medicinal supplement. The at least one phytochemical compound may be selected from the group consisting of racemic and non-racemic equol, resveratrol, and astaxanthin, wherein, the racemic or non-racemic equol forms about 0.1% to 10% of the composition for topical uses, and from about 1 mg to 15 mg for oral uses; wherein resveratrol forms from about 0.1% to 10% of the composition for topical uses, and from about 1 mg to 500 mg for oral uses; and wherein astaxanthin forms about 0.1 to 10% of the composition for topical uses, and from about 1 mg to about 12 mg for oral uses. The at least one other active ingredient may form from about 0.1% to 25% of the composition for topical uses, and from about 1 mg to 2500 mg for oral uses.

Certain further embodiments relate to a method for the prevention and/or treatment of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject, comprising: administering to the subject a composition comprising: (a) one or more polyphenolic compound, its isomer or analog; and (b) one or more other active ingredient selected from the group consisting of collagen, vitamin C, vitamin E, Zinc, grape seed extract, niacinamide, hydroxy acids, hyaluronic acid, and coenzyme Q10. The at least one condition or disorder of skin and accessory structures of the skin may be skin irritation (dermatitis), psoriasis, eczema, skin cancer (melanoma), skin aging—chronological (intrinsic) and photo (extrinsic) aging, augmentation of collagen, elastin, wound healing, facial color and tone, skin smoothness and integrity (barrier repair) including finger nail growth and repair, defense against matrix metalloproteinases, elastase, fibroblast collapse, inflammatory biomarkers, acne, scalp hair loss, thinning of the skin and scalp hair retention; the at least one condition or disorder of musculoskeletal system may be muscle and joint pain, muscle cramps, movement disorder, osteoporosis, and neuromuscular condition; the at least one condition or disorder of the cardiovascular system may be hypertension, heart disease, stroke, tissue hypoxia and blood vessel integrity; the at least one condition or disorder of the inflammatory system may be defending against oxidative stress, oxygen free radicals, inhibition of NFKappB, protecting against oxidative and enhancement of antioxidant protection; the at least one condition or disorder of the neurological system may be memory loss, decreased cognitive function, neurodegenerative disorders (Alzheimer's and Parkinson's disease), white matter brain lesions with aging or trauma, cranial blood vessel integrity and fibromyalgia; the cancer may be skin cancer, DNA mutation, radiation burns, radiation sickness, and premature aging and other tissue and organ cancers; and the at least one condition or disorder of the endocrine/metabolic system may be weight control, hypercholesterolemia, osteoporosis, pituitary disorder, adrenal disorder, pancreatic disorder (diabetes, insulin) and thyroid disorder. The composition may be in a form of lotion, spray, foam, gel, or trans-dermal patch and is administered topically. The composition may be in the form of a liquid, tablet, capsule, dietary or nutritional supplement, and is administered orally. The composition may be administered by an intra-muscular injection or intra-venous methods. The composition may be administered as a food, supplement or OTC product, medicinal food, or medicinal supplement. The may be administered to the subject at a dose of about 0.1% to about 20% by weigh of the composition. The composition may be administered to the subject at a dose of about 0.1 to about 1,500 mg. The composition may be administered to the subject at a dose of about 0.1 to about 500 mg. The composition may be administered to the subject at a dose of about 0.1 to about 100 mg. The composition may be administered to the subject at a dose of about 10 to about 1,500 mg. The composition may be administered to the subject at a dose of about 10 to about 2,500 mg. The at least one phytochemical compound may be selected from the group consisting of racemic equol or non-racemic equol, resveratrol, and astaxanthin.

Yet another embodiment relates to a method for the prevention and/or treatment of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject, comprising: administering to the subject a composition comprising: (a) cannabidiol (CBD); and (b) at least one of: (i) one or more polyphenolic compound, its isomer or analog; and (ii) one or more other active ingredient selected from the group consisting of collagen, vitamin C, vitamin E, zinc, grape seed extract, niacinamide, hydroxy acids, hyaluronic acid, and coenzyme Q10, wherein the CBD binds the CBD receptors and the at least one polyphenolic compound binds the polyphenolic receptors.

### BRIEF DESCRIPTION OF FIGURES

FIG. 1 depicts a chemical structure of THC and CBD.

FIG. 2 depicts the biosynthesis of THC and CBD from cannabigerolic acid (CBGA).

FIG. 3 depicts THC alone, and THC and CDB interactions at the CB1 receptor. THC is a potent partial agonist of CB1. It is this stimulation, which leads to the major psychotropic effects of Cannabis consumption. Right: CBD is a negative allosteric modulator of CB1 receptor, so it changes the shape of the CB1 receptor weakening its ability bind to THC.

FIG. 4 depicts CB Receptor Distribution in the Human Body.

FIG. 5 depicts sites of CBD receptors in skin and immune cells.

FIG. 6 shows the putative receptor targets of CBD.

FIG. 7 shows the chemical structures of CBD and equol (representing all isomeric forms).

FIG. 8 shows the comparison of chemical structures, molecular formulas, molecular weights and C Log P values of 17β-estradiol, resveratrol and equol. C Log P=the log P value of a compound representing its partition coefficient and lipophilicity. The circles at carbon 3 and 17 represent the functional positions for 17β-Estradiol, the circles at carbon 3 and 4′ represent the functional positions for resveratrol and the circles at carbon 7 and 4′ represent the functional positions for equol. (The equol structure shown represents racemic equol, R-equol or S-equol).

FIG. 9 shows examples of polyphenolic molecules.

FIG. 10 depicts the chemical structures and characteristics of astaxanthin and equol. (The racemic equol label represents all isomers of equol along with racemic equol.)

FIG. 11 shows demographics of healthy men in the pain study described herein.

FIG. 12 depicts an example of a short-form McGill Pain Questionnaire.

FIG. 13 shows the results of men's pain study.

FIG. 14 depicts photomicrographs (40× magnification) of full epidermal/dermal thickness human skin cultures. The skin sections were stained with hematoxylin/eosin and all treatment slides displayed intact and healthy epidermal layers (SC=stratum corneum and K=keratinocytes), dermal (F=fibroblasts) components, and epidermal/dermal borders. Standard bar=50 microns.

FIG. 15 shows the validation of full thickness epidermal/dermal human skin cultures cell viability by MTT assay. Testing CBD and equol at various concentration to determine tissue/cell viability as indicated on the y- and x-axis and figure labels for subsequent human skin gene analysis. Triton X-100 treatment (cytotoxic control). Note: The MTT assay is a colorimetric assay, which has been validated for measuring cell metabolic activity. It is based on the ability of nicotinamide adenine dinucleotide phosphate (NADPH)-dependent cellular oxidoreductase enzymes to reduce the tetrazolium dye MTT to its insoluble formazan, which has a purple color. The absorbance of this colored solution can be quantified by measuring at a certain wavelength (usually between 500 and 600 nm) by a spectrophotometer. The MTT method is one of the most widely used methods to analyze cell proliferation and viability in human skin cells (Triglia et al., 1991).

FIG. 16 shows the validation of the dermal layer (only) of the human skin cultures viability by MTT assay (see details above in FIG. 15 legend for the MTT assay). Testing CBD and equol at various concentration to determine tissue/cell viability as indicated on the y- and x-axis and figure labels for subsequent human skin gene analysis. Triton X-100 treatment (cytotoxic control).

### DETAILED DESCRIPTION

While the present invention may be embodied in many different forms, for the purpose of promoting an understanding of the principles of the present invention, reference will now be made to embodiments, some of which are illustrated in the drawings, and specific language will be used to describe the same. It will nevertheless be understood that no limitation of the scope of the invention is thereby intended. Any alterations and further modifications in the described embodiments and any further applications of the principles of the present invention as described herein are contemplated as would normally occur to one skilled in the art to which the invention relates. Additionally, in the detailed description below, numerous alternatives are given for various features related to the structure or composition of materials, or to modes of carrying out methods. It will be understood that each such disclosed alternative, or combinations of such disclosed alternatives, can be combined with the more generalized features discussed in the Summary above, or set forth in the Listing of Certain Embodiments below, to provide additional disclosed embodiments herein.

### Definitions

In the present application, the following terms are used throughout and are defined for the purposes of this application as follows:

The article “a” or “an” as used herein means “one or more” unless otherwise specified.

The term “or” can be conjunctive or disjunctive.

Terms such as “include,” “including,” “contain,” “containing,” “have,” “having,” and the like mean “comprise” or “comprising.”

The term “comprising” means that other steps and other ingredients which do not affect the end result can be added. This term encompasses the terms “consisting of” and “consisting essentially of.”

The term “at least one,” as used herein, means one or more and thus includes individual components as well as mixtures or combinations.

Herein, “mixtures” is meant to include a simple combination of materials and any compounds that may result from their combination.

The terms “derived from” or “produced from” as used herein, unless otherwise specified, indicate that a particular thing (e.g., chemical compound) or group of things has originated from the source specified, but has not necessarily been obtained directly from the specified source.

The term “derivative” refers to a compound that is derived from a similar or parent compound by a chemical reaction, e.g., by the addition of a side group or substitutive chemical group or chemical modification of at a particular atom in the compound.

The term “isomer” refers to one of two compounds with the same chemical formula, but a different arrangement of atoms in space in the molecule and each having different properties. For example, isomers can be represented by left-handed and right-handed gloves, which are not superimposable upon one another. Also, types of stereoisomers consist of enantiomers, which are mirror-images which contain chiral centers and are not superimposable, such as left-handed versus right-handed gloves, such as isomers of equol, i.e., S-equol and R-equol.

The term “analog” refers to a compound with a molecular structure closely similar to that of another or parent compound.

The terms “prevention” or “preventing” as used herein refer to treating or providing relief from a disease or health condition from occurring, as to improve health or treat the condition, levels or symptoms of disease or discomfort from a health condition or physiological or morphological state.

The terms “treatment” or “treating” refer to improvement in a subject's condition, or the activity of making an effort to correct, or at least make more acceptable, conditions that are disconcerting, difficult or challenging to endure related to subject's health condition(s), such as anatomical morphology, aging, pain, inflammation, neural degeneration, cognition, memory, neural function, physical appearance, biological functions, or discomfort from such anatomical, morphological or physiological states of being.

The terms “ameliorate” or “amelioration” refer to making something better or more tolerable in a subject's health condition(s) such as the process of making a bad, negative or unpleasant situation or state better, enhanced or healthier including the physical appearance, anatomical, morphological, mental, neurological, emotional, homeostasis of a cell, tissue or organ, and the biological states of being.

The terms “composition” and “formulation” are used herein interchangeably and refer to a mixture of components formulated for administration (topical, oral, etc.) to a mammalian subject.

The term “skin and accessory structures of the skin” refers to the natural outer covering of the body of a person or animal, including any accessory structures of the skin, such as hair, nails, sweat glands, and sebaceous glands.

Described herein are compositions including a polyphenolic compound, its isomer or analog, and one or more other active ingredient selected from the group consisting of collagen, vitamin C, vitamin E, zinc, grape seed extract, niacinamide, hydroxy acids, hyaluronic acid, and coenzyme QI for the prevention or treatment of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject. Also, described herein are compositions including cannabidiol (CBD) and at least one of: one or more polyphenolic compound, its isomer or analog; and one or more other active ingredient selected from the group consisting of collagen, vitamin C, vitamin E, zinc, grape seed extract, niacinamide, hydroxy acids, hyaluronic acid, and coenzyme Q10. Furthermore, described herein are methods of using the compositions for the prevention or treatment of the at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject.

Various applications include cosmetic and pharmaceutical treatments for the public domain and in medical/dental, veterinarian, environmental work conditions. Notably, CBD's and polyphenol(s)'s actions (along with other phytochemicals and natural products) are due to their powerful antioxidant, anti-inflammatory, activation of Nrf2 defense system mechanisms, inhibition of NFkapp B, and protection of cells and tissues by inhibiting oxidative stress, enhancement of DNA methylation, and thereby decreasing damage, and enhancing binding to estrogen receptor subtypes, 5alpha-dihydrotestosterone (DHT), and the binding and activation of estrogen related receptor gamma (γ) that inhibits neoplastic growth. In fact, the combination of CBD and polyphenol(s) administered together or in combination with other natural active ingredients, such as collagen, vitamin C, vitamin E, zinc, grape seed extract, niacinamide, hydroxy acids, hyaluronic acid or coenzyme Q10 displayed surprising results, which were not seen when the test materials were administered alone, which provide evidence for their synergistic, pleiotropic and entourage effects on various biological factors, functions and health improving actions.

I. Compositions

In one embodiment, described herein is a composition for the prevention and/or treatment of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject.

The composition includes at least two of the following ingredients (a) through (c):

(a) cannabidiol (CBD), analogs and/or derivatives thereof;
(b) a polyphenolic compound, isomers, and/or derivatives thereof;
(c) and at least one other active ingredient, such as, e.g., collagen, vitamin C, vitamin E, zinc, grape seed extract, niacinamide, hydroxy acids, hyaluronic acid, and coenzyme Q10.

In certain further embodiments, the composition includes (a) through (c) as follows:

(a) cannabidiol (CBD), isomers, analogs and/or derivatives thereof;
(b) a polyphenolic compound, isomers, analogs, and/or derivatives thereof;
(c) and at least one other active ingredient, such as, e.g., collagen, vitamin C, vitamin E, zinc, grape seed extract, niacinamide, hydroxy acids, hyaluronic acid, and coenzyme Q10.

One embodiment relates to a composition including a polyphenolic compound, its isomer or analog, and one or more other active ingredient selected from the group consisting of collagen, vitamin C, vitamin E, zinc, grape seed extract, niacinamide, hydroxy acids, hyaluronic acid, and coenzyme QI for the prevention or treatment of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject.

Another embodiment relates to a composition including a combination of CBD and at least one of: one or more polyphenolic compound, its isomer, or analog; and one or more other active ingredient (its isomer, derivative or analog) selected from the group consisting of collagen, vitamin C, vitamin E, zinc, grape seed extract, niacinamide, hydroxy acids, hyaluronic acid, and coenzyme QI for the prevention and/or treatment of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject.

In one embodiment, the combination includes CBD and at least one polyphenolic molecule (e.g., racemic equol or non-racemic equol, or other phytochemical(s)), along with at least one other natural active ingredient is formulated for use as a topical (lotion, spray, trans-dermal patch), oral (tablet, liquid, capsule), injectable, pharmacological formulation or an over-the-counter (OTC) agent for the prevention and/or treatment of at least one of the following conditions of: a) skin and accessory structures of the skin, b) musculoskeletal system, c) cardiovascular system, d) inflammatory system, e) neurological system, f) cancer, g) endocrine/metabolic conditions, and other biological factors and functions.

In certain embodiments, different percentages or combinations of the described composition components may include isomers, analogs or derivatives of the original molecules and other natural active ingredients described herein formulated as a topical (lotion, spray, trans-dermal patch), oral (tablet, liquid, capsule), injectable, pharmacological formulation or OTC agent. These formulations may be used for the prevention and/or treatment of at least one condition of: a) skin and accessory structures of the skin, b) musculoskeletal system, c) cardiovascular system, d) inflammatory system, e) neurological system, f) cancer, g) endocrine/metabolic system, and other biological factors and functions.

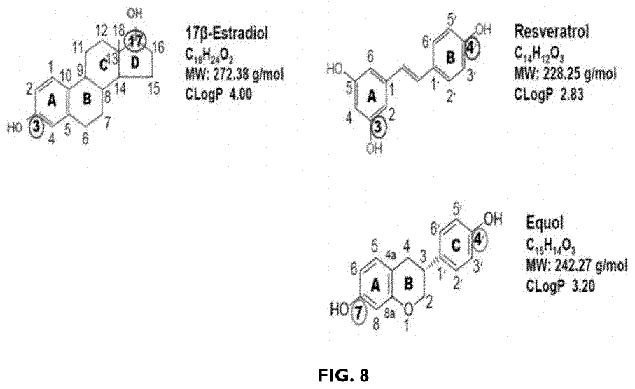

A further embodiment relates to a composition including CBD in combination with a polyphenolic compound with chemical modifications such as hydroxyl, methyl, butyl, alcohols, esters, ketones, acetates, phosphates at carbon number 7, 2′, 3′, 4′ or 5′ (FIG. 8) or with other combinations of analogs (as an example) along with natural other active ingredients formulated for a topical (lotion, spray, trans-dermal patch), oral (tablet, liquid, capsule), injectable, pharmacological administration or as an OTC agent for the prevention and/or treatment of at least one condition of: a) skin and accessory structured of the skin, b) musculoskeletal system, c) cardiovascular system, d) inflammatory system, e) neurological system, f) cancer, g) endocrine/metabolic system, and other biological factors and functions. Notably, the resveratrol analog, 4′-acetoxy resveratrol (4AR) was used and reported herein, which is an ester of resveratrol (Table 22).

Another embodiment relates to a composition including CBD and a different combinations of polyphenolic compounds (e.g., equol analogs, resveratrol, etc.) along with other natural active ingredients, formulated as a topical (lotion, spray, trans-dermal patch), oral (tablet, liquid, capsule), injectable, pharmacological formulation or an OTC agent for the prevention and/or treatment of at least one condition of: a) skin and the associated structures of the skin, b) musculoskeletal system, c) cardiovascular system, d) inflammatory system, e) neurological system, f) cancer, g) endocrine/metabolic system, and other biological factors and functions.

Another embodiment relates to a composition formulated as a food, dietary supplement or fragrance product to deliver CBD, and/or polyphenolic compound (e.g., equol (racemic or non-racemic), equol analogs, other polyphenolic compounds, such as resveratrol) along with other natural active ingredients for the prevention and/or treatment of at least one condition of: a) skin and associated structures of the skin, b) musculoskeletal system, c) cardiovascular system, d) inflammatory system, e) neurological system, f) cancer, g) endocrine/metabolic system, and other biological factors.

In the described embodiments, the dose of any combination of CBD and/or polyphenolic/phytochemical isomers, analogs along with other natural active ingredients may be between 0.01 mg to 1500 mg or 0.01% to 30% depending upon the body weight and expected, calculated or known method of treatment. Broadly covering all ingredients, dosing representing various embodiments for topical applications may include: 1 mg/ml to 20 mg/ml, or 0.1% to 8%, or 1% to 20% whereas, for oral dosing of various embodiments may include: 1 mg to 10 mg, or 10 mg to 800 mg or 50 mg to 1,200 mg. Any dosage amount or percentages in-between the above amounts are also contemplated.

A. Cannabidiol (CBD)

In certain embodiments, the described compositions may include CBD.

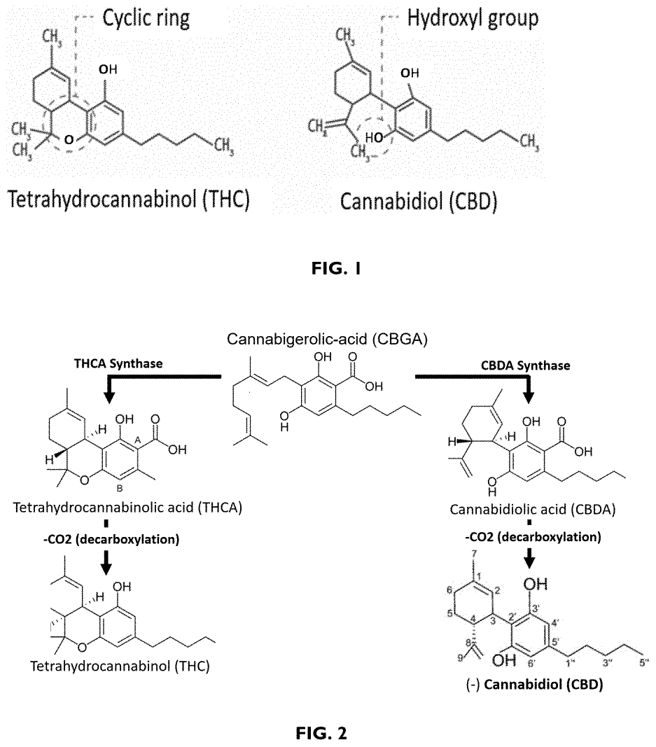

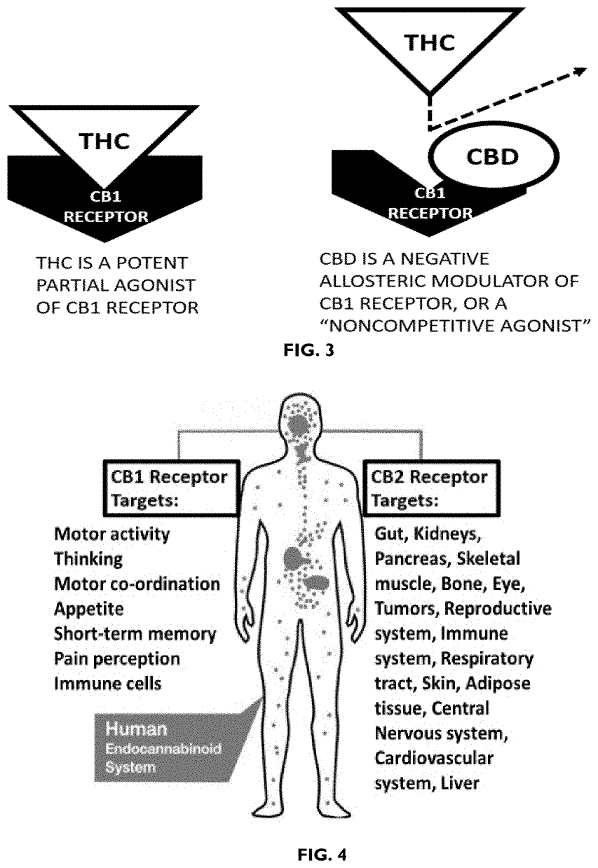

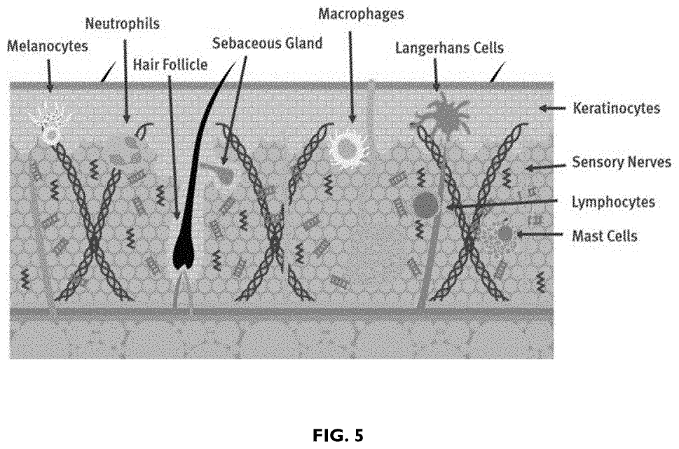

CBD was first isolated in 1940 by Roger Adams at the University of Illinois, while THC was isolated in 1964 by Raphael Mechoulam. At the most fundamental level, THC and CBD are different because of their differing physiological effects. CBD is non-psychotropic, and therefore, does not illicit a “high,” whereas, THC is psychotropic and is the only known Cannabis-derived compound to illicit a “high” (Rong et al., 2017; Burstein, 2019). Shown in FIG. 1 are some of the key differences and similarities between CBD and THC chemical structures.

Both CBD and THC share the same molecular formula, C₂₁H₃₀O₂, containing twenty-one atoms of carbon, thirty of hydrogen and two of oxygen. Their molecular mass is practically identical with THC and CBD having masses of 314.469 g/mol 314.464 g/mol, respectively (Rong et al., 2017; Burstein, 2019).

The biosynthesis of THC and CBD in Cannabis also follows a very similar pathway (see FIG. 2). Cannabigerolic acid (CBGA), the precursor to all-natural cannabinoids, is cyclized into tetrahydrocannabinolic acid (THCA) and cannabidiolic acid (CBDA) by THCA and CBDA synthase, respectively. The final products of THC and CBD are formed via decarboxylation of these acidic forms. Structurally, however, there is one important difference. Where THC contains a cyclic ring (see FIG. 1), CBD contains a hydroxyl group. It is this seemingly small difference in molecular structure that gives the two compounds entirely different pharmacological properties (Pisanti et al., 2017; Rong et al., 2017; Burstein, 2019).

CBD can be extracted from hemp. Hemp is rich in many phytocannabinoids, including CBD. In addition to phytocannabinoids, the hemp plant also contains many other beneficial phytonutrients like fatty acids, plant sterols, terpenes, chlorophyll and naturally occurring vitamin E (Rong et al. 2017; Pollastro et al., 2018; Burstein, 2019; Franco & Perucca, 2019).

When CBD is extracted from hemp, other plant components come along with it. This is what is referred to as a “whole plant” or “full spectrum” hemp extract. For example, other phytocannabinoids or other minor cannabinoids present in hemp include cannabigerol (CBG), cannabinol (CBN), and cannabichromene (CBC).

Many CBD products are made using a full spectrum, whole plant CO₂ extraction method. The basic and biggest difference is that broad-spectrum does not contain THC, whereas the full spectrum does.

To isolate CBD only, analytical chemical methods, such as HPLC can be used where a “fraction” or isolated “peak” of CBD is taken from the extracted material. However, because CBD and THC share the exact same molecular formula, C₂₁H₃₀O₂, and very similar chemical masses or weights (THC and CBD having masses of 314.469 g/mol 314.464 g/mol, respectively), even the isolated “fractions” obtained by analytical chemical methods are likely to have a small percentage of THC in the “purified” CBD material (Pisanti et al., 2017; Hlozek et al., 2017). Therefore, further isolation and purification steps must be performed to obtain high purity CBD material.

A high purity CBD may also be purchased from commercial sources, such as a) Extract Labs, Boulder, Colo., USA, b) Cayman Chemical, Ann Arbor, Mich., USA, or c) Charkit Chemical Company LLC, Norwalk, Conn., USA.

As with many of the cannabinoids, THC and CBD have low solubility in water, but good solubility in most organic solvents, particularly lipids and alcohols. Both THC and CBD are present in Cannabis in a mixture of acidic forms, which are readily de-carboxylated and chemically altered upon heating.

In the described compositions, CBD may form anywhere from 0.01 to 99% by weight/weight of the composition. For example, preferably CBD forms at least 1% w/w of the composition; more preferably, at least 5% w/w of the composition; more preferably; at least 10% w/w of the composition; more preferably, at least 15% w/w of the composition; more preferably, at least 20% w/w of the composition; more preferably at least 25% w/w of the composition; more preferably, at least 30% w/w of the composition; more preferably, at least 30% w/w of the composition; more preferably, at least 35% w/w of the composition; more preferably, at least 40% w/w of the composition; more preferably, at least 45% w/w of the composition; more preferably at least 50% or more w/w of the composition. Any dosage amount or percentages in-between the above amounts are also contemplated.

B. Polyphenolic Compound(s)

The described composition may include at least one polyphenolic compound, or isomer, and/or derivative thereof.

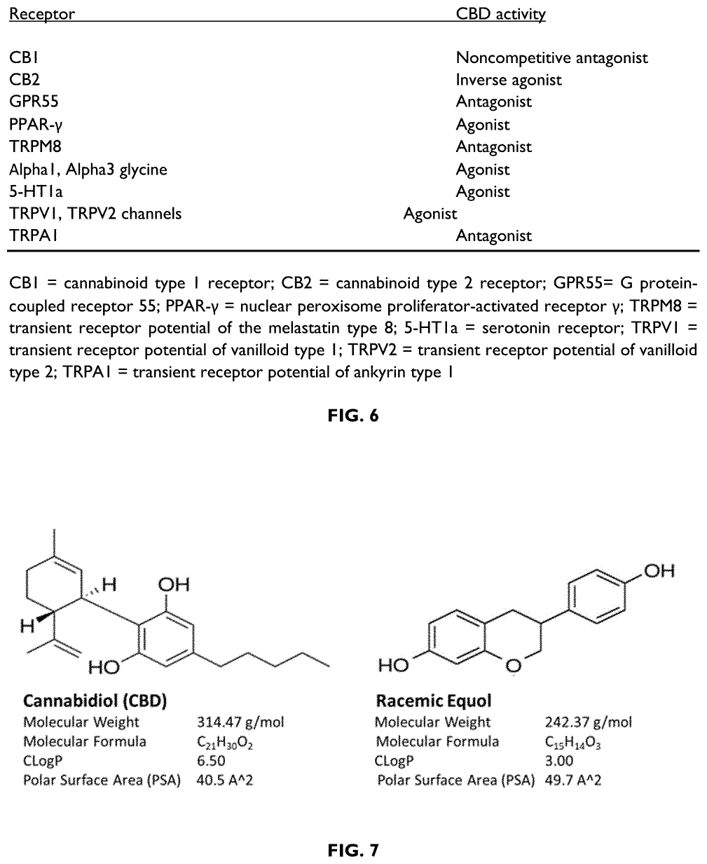

The term “polyphenolic compound” refers to any natural, but also synthetic or semisynthetic, organic chemical characterized by the presence of large multiples of phenol structural units. The number and characteristics of these phenol structures underlie the unique physical, chemical, and biological (metabolic, toxic, therapeutic, etc.) properties of the particular compound. Phenolics also have at least one hydroxyl group attached to an aromatic ring. Examples of polyphenolic compounds include, but are not limited to isoflavonoids, such as equol (racemic and non-racemic), daidzein, genistein, and other phytochemicals, resveratrol, astaxanthin, lignans, phenolic acids, caffeic acids. The chemical structure of equol is shown in FIG. 7 and FIG. 8.

Equol

In certain embodiments, the described composition includes a phytochemical, such as equol. Equol is an isoflavonoid (has two phenolic rings with hydroxyl groups on each ring that provide functional points for biological activity (Lephart, 2013b; Lephart, 2016)) and a phytoestrogen (has a selective estrogen receptor modulator (SERM) characteristics that yield an enhanced/sustained delivery into the dermal skin layers (Gopaul et al., 2012; Lephart 2013b; Lephart 2016; Lephart, 2017; Lephart, 2018a) which inhibits dermal aging (Gopaul et al., 2012; Lephart, 2016; Lephart, 2018b; Lephart, 2019)).

While endogenous estrogenic hormones such as 17β-estradiol are steroids, with a cyclo-hexane-phenantrene parent chemical structure that is derived from cholesterol, equol is not a steroid (Lephart, 2016). However, equol and 17β-estradiol have similar chemical structures/confirmations and molecular weights (C₁₅H₁₄O₃ vs. C₁₈H₂₄O₂; 242.3 g/mol vs. 272.4 g/mol, respectively) (Lephart, 2016; see FIG. 8).

Equol has a chiral center at carbon 3, and thus can exist in two mirror image forms known as enantiomers (S-equol and R-equol) (Lephart, 2013b; Setchell and Clerici, 2010, see FIG. 8). The term “racemic” refers to the exact equal portions of isomers or a racemic mixture, or racemate, is one that has equal amounts of left- and right-handed enantiomers of a chiral molecule (Racemic, 2020). Equol can be produced as racemic equol with presumably, extract equal amounts of S-equol and R-equol (Lephart, 2013b; Lephart 2016). However, in man and animals only S-equol is produced by intestinal bacteria conversion from daidzein, and some individuals have the ability of producing higher levels of equol than others (Lephart, 2013b, Setchell and Clerici, 2010; Setchell et al., 2005). These individuals have been identified as “equol producer” (Lephart, 2013b, Setchell and Clerici, 2010). The term “equol producer” is a descriptive or arbitrary term for humans that maintain S-equol levels around or above 10 to 20 ng/ml after consumption of soy food products that infers protective health benefits (Lephart, 2013b; Lephart, 2016). S-Equol can be found in plant products such as beans, cabbage, lettuces, tofu and other food and animal products (Abiru et al., 2012; Common and Ainsworth, 1961; Hounsome et al., 2009; Hounsome et al., 2010; Jou et al., 2013).

Both equol isomers and racemic equol exhibit antioxidant, anti-inflammatory, skin protectant (against ROS/oxidative stress) and specifically anti-androgen hormonal actions by binding free 5α-dihydrotestosterone (5α-DHT) as a selective androgen modulator (SAM) (Gopaul et al., 2012; Lephart, 2013a; Lephart, 2016; Lephart, 2017; Lund et al., 2004; Lund et al., 2011; Magnet et al., 2017; Oyama et al., 2012). A comparison among R-equol, racemic equol and S-equol for various biochemical characteristics and molecular/biomarker parameters is shown in Table 1.

TABLE 1
Comparison of R-Equol, Racemic Equol and S-Equol for Various Characteristics/Parameters:

| Characteristic/Parameter | R-Equol | Racemic Equol | S-Equol |
|---|---|---|---|
| 1. Binds 5α-DHT | Yes | Yes | Yes |
| 2. Inhibits 5α-Reductase | Yes | Yes | No |
| Enzyme (type 1 in skin) | | | |
| 3. Binds Estrogen Receptor | Ki = 15.4 nM | IC₅₀ = 0.2 μM | Ki = 6.4 nM |
| Beta (β) | | | |
| 4. Binds Estrogen Receptor | Ki = 27.4 nM | IC₅₀ = 1.5 μM | Ki = 15.4 nM |
| Alpha (α) | | | |
| 5. Binds/Activates Estrogen | Not Determined | Yes | Not Determined |
| Related Receptor gamma (γ) | | | |
| 6. Present in plants/food | fermented plants | Not Determined | plants/tofu/eggs/milk |
| products | | | |
| 7. GI Intestinal Metabolite | Not Determined | Not in part | Yes-from daidzein |
| (humans) | | | |
| 8. Oral Metabolism in Humans | Similar | Similar | Similar |
| (pharmacokinetics compared | | | |
| to each other) | | | |
| 9. Stimulates Collagen | Yes | Yes | Yes |
| 10. Stimulates Elastin | Yes | Yes | No |
| 11. Inhibits Elastase | Not Determined | Yes | Not Determined |
| 12. Stimulates TIMPI | No, or at very low | Yes | Yes |
| 13. Inhibits MMPs | Yes, to high levels of | Yes, to moderate | Yes, to low levels of |
| | inhibition | levels of inhibition | inhibition |
| 14. Stimulates Growth Factors | Yes, to high levels | Yes, to moderate | Yes, to low levels |
| | | levels | |
| 15. Strong Antioxidant | Yes, to high levels | Yes, to moderate | Yes, to moderate |
| | | levels | levels |
| 16. Binds Nrf2/activates other | Not Determined | Yes | Not Determined |
| antioxidants | | | |
| 17. Inhibits NFkappB | Not Determined | Yes, high inhibition | Not Determined |
| 18. Inhibits Inflammatory | Yes, high inhibition | Yes, high inhibition | Yes, low inhibition |
| Molecules | | | |

Legend to Table 1:
5alpha-dihydrotestosterone (5α-DHT);
GI = gastrointestinal;
TIMP1 = tissue inhibitor of matrix metalloproteinase 1
MMP = matrix metalloproteinase which breaks down collagen and elastin

As shown, S-equol binds estrogen receptor (ER) beta (β) approximately ⅕ as well as the natural endogenous steroid hormone, 17β-estradiol, while S-equol has low affinity for ER alpha (a). Conversely, R-equol has weak affinity for either ERα or ERβ, and in general, has weak estrogenic properties at best.

Specifically, equol when applied to human skin has a sustained release mechanism from the epidermis (where equol binds to estrogen receptor beta that is abundant in keratinocytes) into the dermal tissue layers (Lephart, 2013a). For example, a single dose of topically applied equol has a unique skin penetration profile that peaks around 8 hours, then has a sustained released for up to 28 hours after the initial application (Lephart, 2013a, Lephart, 2016).

The lipophilic nature of equol is shown via its octanol-water partition coefficient of 3.0 to 3.2, which is higher than other polyphenolic molecules (Rothwell et al., 2005), see FIG. 7 and/or FIG. 8. For example, the octanol-water partition coefficients for resveratrol=3.1; genistein=3.0 and diadzein=2.5. Additionally, intestinal absorption data supports this proposition where equol (either as the R- or S-isomer) has the highest absorption levels (80 to 85%) compared to other isoflavonoids, like genistein (at 15-20%) or daidzein (at 30-40%) (Setchell and Clerici, 2010; Setchell et al., 2009). Drug delivery studies have shown the more stable isomer is R-equol vs. S-equol (Alvira et al., 2008), and in vitro culture data suggests that the R-isomer accounts for the chemo-protective effects of equol rather than the S-isomer in breast and prostate cancer cells (Magee et al., 2006).

Equol has powerful antioxidant activity and its unique molecular and biochemical messenger properties with implications in treating age-related diseases (Lephart 2013a; Lephart 2013b; Setchell and Clerici, 2010).

Comparative studies examining polyphenolic compounds demonstrated that equol is a superior antioxidant, having greater antioxidant capacity than, e.g., vitamin C or vitamin E in several in vitro tests (Arora et al., 1998; Mitchell et al., 1998). In in one study, equol exhibited one of the highest antioxidant activities, when three different in vitro assays were used, and equol was more effective than the positive controls quercetin and ascorbic acid (Rufer and Kulling, 2006). Finally, equol has greater antioxidant activity (i.e., protection against oxidative damage to lipid membranes, etc.) compared to genistein (Mitchell et al., 1998; Rufer and Kulling, 2006).

Oxidative stress via radical oxygen species (ROS) production by UV-light-induced signal cascades is known to cause skin aging and the pathology of skin disease (Bickers and Athar, 2006; Khol et al., 2011; Naylor et al., 2011). Nuclear-factor-erythroid 2-related factor 2 (Nrf2) is a master regulator of the transcriptional response to oxidative stress, and it is structurally and functionally conserved from insects to humans (Lacher et al., 2015). Nrf2 plays a key role in the cellular defense against oxidative and xenobiotic stressors by its capacity to induce the expression of numerous genes, which encode detoxifying enzymes and antioxidant proteins that provide protection in endothelia cells, skin morphogenesis, wound repair and skin cancer (Beyer et al., 2007; Greenwald et al., 2015; Sekhar and Freeman, 2015; Zhang et al., 2013). Additionally, Nrf2 deficiency or Nrf2-knockdown is known to cause lipid oxidation, inflammation, and extra cellular matrix-protease expression [e.g., MMPs, cyclo-oxygenases (Cox)] in UVA-irradiated skin fibroblasts or heat shock-induced human dermal fibroblasts (Gruber et al., 2015; Park and Oh, 2015).

Bottai et al. (2012) reported that 17β-estradiol protects human keratinocytes and fibroblasts against oxidative damage by counteracting hydrogen peroxide-mediated lipoperoxidation. Furthermore, racemic equol has been shown to increase nuclear-factor-erythroid 2-related factor 2 (Nrf2) (Lephart, 2016).

As discussed by Jackson et al. (2014) the molecular mechanism involves the release of Nrf2 (transcription factor) from Keap 1, its cytoplasmic binding protein and subsequent binding to the antioxidant response element (ARE) that is present in the promoter region of genes for antioxidant proteins and enzymes. Specifically, equol may increase Nrf2 levels and/or bind to the estrogen-responsive elements (EREs) in the promoter region the Nrf2 gene and/or increase gene expression of other antioxidant genes. In support of this concept, Zhang et al. (2013) showed that S-equol provided protection against peroxide-induced endothelial cell apoptosis by activation of estrogen receptor and Nrf2/ARE signaling pathways. Finally, Froyen and Steinberg (2011) reported that racemic equol increased the expression of the xenobiotic metabolizing enzyme quinone reductase (both mRNA and protein levels) via similar molecular mechanisms involving ER β and Nrf2.

In addition to the above, Gegotek et al., in 2019, in the journal Cells showed the differences in the proteome (i.e., the proteome is the entire set of proteins that is, or can be, expressed by a genome, cell, tissue, or organism at a certain time) profile of CBD-treated human skin fibroblasts following UVA or UVB irradiation in 2D and 3D cell cultures. The main findings of this study showed that in 2D cultured cells, following UV radiation, the major changes were associated with proteins involved in antioxidant formation and combating the inflammation response, while in the 3D cultures fibroblasts, CBD action against UV induced changes were mainly associated with the activation of signaling pathways demonstrating cell to cell interactions and skin cell metabolism. Thus, this report showed that: a) CBD decreased the UV-induced expression of metalloproteinases, thus preventing damage in the intracellular matrix, b) CBD participates in the anti-inflammatory signaling pathway by inhibiting NFkappB and c) CBD significantly enhanced the level of enzymes related to glutathione-related metabolism such as γ-glutamylocysteine synthetase (γ-GCS GSH), GSH-S-transferase (GST), and glutathione peroxidase (GSH-Px), which are part of the antioxidant response, among many other alterations where CBD enhanced skin health (Gegotek et al., 2019 Cells).

ROS production is known to damage DNA, proteins and lipids and other cellular components (Bickers and Athar, 2006; Gonzaga, 2009; Khol et al., 2011). In human skin an important aging biomarker is proliferating cell nuclear antigen (PCNA) involved in DNA repair and it is known to decrease in aged skin (Goukassian et al., 2000; Takahashi et al., 2005). Racemic equol was shown to stimulate the gene expression of PCNA that is known for its protective skin effects (Gopaul et al., 2012; Lephart, 2013a).

Several reports have shown that nerve growth factor (NGF) is important in cell survival, wound healing by stimulation of collagen and regeneration of cutaneous nerves (Marconi et al., 2003; Nithy et al., 2003; Yaar et al., 1991; Zhai et al., 1996). It is known that NGF expressed in keratinocytes is reduced by UVB exposure (by sun/photo-aging) (Marconi et al., 2003). Gopaul et al. (2012) and Lephart (2013a) showed that racemic equol stimulated NGF gene expression in human skin that suggested protective dermal effects by NGF from oxidative stress.

AP-1 is a nuclear transcription element that is part of the oxidative stress cascade. It induces MMPs in human skin that in turn activates collagen degradation (Hung et al., 1997). AP-1 also blocks the positive pro-collagen actions of transforming growth factor-β1 (Fischer et al., 1996). Kang et al (2007a) reported that the anti-tumor effects of racemic equol are due to the inhibition of cell transformation by the MEK signaling pathway by blocking AP-1. Racemic equol dose-dependently attenuated TPA-induced activation of AP-1, whereas daidzein did not exert any effect when tested at the same concentrations (Kang et al., 2007a). Finally, ER β signaling has been shown to protect against transplanted skin tumor growth in mice (Cho et al., 2010), which implicated a common mechanism of how the blocked AP-1 actions by equol reported by Kang et al. (2007a) may be mediated.

Additionally, equol has been shown to bind to ERR γ that in turn enhanced the transcriptional activity of ERR γ, which is known to protect against neoplastic growth (Hirvonen et al., 2011). Equol's precursor molecule daidzein did not have this effect (Krah-Bertil et al., 2008). It is also known that ERR γ is present in keratinocytes and fibroblasts in human skin where its actions may show similar favorable protection as that seen against breast and prostate cancers (Hirvonen et al., 2011; Krah-Bertil et al., 2008)

It has been demonstrated that ER β signaling in the prostate (and breast tissue) are associated with decreased inflammation and neo-plastic growth (Lephart, 2014). S-Equol is known to have a high affinity for ER β, which is the predominate ER in keratinocytes and fibroblasts in human skin (Pelletier and Ren, 2004; Setchell et al., 2005; Thornton et al., 2003) and this ER signaling mechanism protects against immune suppression by UV radiation exposure (Chang et al., 2010; Pomari et al., 2015; Widyarini et al., 2006a). The high affinity for ER β by S-equol may account for the unique topical dermal absorptive and ‘reservoir’ penetration method into human skin that may account, in part, for its positive benefits (Lephart, 2013a).

There is further evidence that this protective effect may be mediated via estrogen receptor beta not only in breast and prostate cancers but neurodegenerative disorders like Alzheimer's disease as well (Chiou et al., 2018; Yao et al., 2013; Lephart, 2014).

Finally, in this regard pertaining to neuroprotection, equol has been shown to attenuate microglial activation and protect neurons from neuroinflammatory injury by the downregulation of neuronal apoptosis along with increased neurite outgrowth via neurtrophins like nerve growth factor (NFG) from in vitro studies (Subedi et al., 2017).

In 2013, Pucci et al., studied the epigenetic control of skin differentiation genes by phytocannabinoids in the British Journal of Pharmacology. In general, the key results showed that CBD significantly reduced the expression of all the genes tested in differentiated HaCaT cells, by increasing DNA methylation of keratin 10 gene through a CB1 receptor-dependent mechanism. In addition, CBD increased global DNA methylation levels by selectively enhancing DNMTI expression, which suggests that CBD is a transcriptional repressor that can control cell proliferation and differentiation that may have potential as a novel therapeutic agent for various skin diseases, most notably cancer.

The pro-inflammatory transcription factor NFkappa B has been studied for more than 30 years. It was first discovered by Sen and Baltimore in 1986 (Gupta et al., 2010). NF-kappa B remains an exciting and active area of investigation, where it is expressed in all cell types and is evolutionarily conserved (Ghosh et al., 1998). NFkappa B is involved in the oxidative stress mechanism by the expression of numerous genes such as the cytokines and plays a major role in the pathology of inflammatory disease (Gilmore, 2006; Gosh et al., 1998; Hayden and Gosh, 2012; Schreck et al., 1991).

Several investigators have examined the inhibition of NFkapp B by equol. In (2005), Kang et al. showed that racemic equol inhibited tumor necrosis factor-α gene expression by blocking NFkapp B in mouse macrophages that was independent of an estrogen receptor mechanism. Tumor necrosis factor-α is a cell signaling protein (cytokine) involved in systemic inflammation (Kang et al., 2005). Furthermore, Kang et al. in (2007b) reported the dose-dependent inhibitory effects of racemic equol (by in vivo administration) on nitric oxide (NO) production and inducible nitric oxide synthase (iNOS) gene expression in murine macrophages from lipopolysaccharide (LPS)-treated mice. The enzyme NO synthase (NOS) generates NO that is a free radical and the overproduction of NO by iNOS is associated with the development of several diseases including atherosclerosis, stroke, septic shock and Alzheimer's disease (Kang et al., 2007b). The gene expression of iNOS is regulated mainly at the transcriptional level in macrophages and NFkappa B via the MAPK pathway and PI3K/Akt pathways. The MAPK pathway has been well known to be involved in the regulation of iNOS gene expression and NF-kappa B activation (Kang et al., 2007b). In this study, LPS-induced activation of Akt was suppressed by racemic equol, and it also blocked LPS-induced NFkappa B activation.

Polyphenols (such as equol and resveratrol) and CBD are known to activate Nrf2=Nuclear-factor-erythroid 2-related factor 2 that is a master regulator of the transcriptional response to oxidative stress; it plays a key role in the cellular defense against oxidative and xenobiotic stressors by its capacity to induce the expression of numerous genes, which encode detoxifying enzymes and antioxidant proteins. Also, CBD and equol have been shown to inhibit NFkappa B as described in the previous material presented above (Kang et al., 2005). For example, As early as 1998, Hampson et al., reported in the Proceedings of the National Academy of Science (USA) that CBD was a powerful antioxidant, which provided neuroprotective effects that were greater than the dietary antioxidants, alpha-tocopherol or ascorbate (Hampson et al., 1998).

Also, equol has been shown to inhibit NFkappa B=is a pro-inflammatory transcription factor NFkappa B that is involved in oxidative stress mechanisms by the expression of numerous genes such as cytokines and plays a major role in the pathology of inflammatory diseases; Equol also inhibits AP-1 and neoplastic cell growth via estrogen related receptor gamma (γ) and protects DNA and enhances tissue and nerve repair (Lephart, 2016; Casares et al., 2019).

Extensive research during the last two decades has revealed the mechanism by which continued oxidative stress can lead to chronic inflammation, which in turn, could mediate most chronic diseases including cancer and skin damage (Bar-Or et al., 2015; Bickers and Athar, 2006; Droge, 2002; Maes et al., 2011; Maritim et al., 2003; Valko et al., 2007). Recent research on polyphenolic molecules from plants sources has expanded the horizon for potential therapeutic remedies and treatments (Evans and Johnson, 2010; Adlercreutz et al., 2004; Park and Pezzuto, 2015). It is well documented that various pro-inflammatory markers in human skin are increased with UV exposure (Bickers and Athar, 2006; Khol et al., 2011; Strickland et al., 1997).

It has been shown that racemic equol inhibited the gene expression of several pro-inflammatory biomarkers such as interleukin-1 alpha (IL-1A), IL-6, IL-8 and interleukin-1 receptor 2 as well as COX-1 and tumor necrosis factor receptor (Gopaul et al., 2012, Lephart, 2013a; Lephart, 2019). These pro-inflammatory biomarkers are known to increase with UV exposure and aging (Bickers-Athar, 2006; Natarajan et al., 2014; Strickland et al., 1997).

It is also known that ER β signaling protects epidermal cytokine expression and immune function by UVB exposure (Chang et al., 2010; Cho et al., 2008; Widyarini et al., 2012; Widyarini et al., 2006a). Since racemic equol has been utilized in most research investigations, the S-equol portion in racemic equol may act by binding to ER β receptors in keratinocytes. This may explain the obtained gene expression results, where equol inhibited several pro-inflammatory biomarkers (Gopaul et al., 2012). However, a comprehensive study that examined the equol isomers along with racemic equol in human skin via gene array analysis suggested that R-equol and/or racemic equol are better inhibitors of the pro-inflammatory biomarkers (Lephart, 2013a).

As described previously, CBD protects against UVA and UVB radiation by: 1) inhibiting matrix metalloproteinases, 2) activating anti-inflammatory mechanism by blocking NFKappB (i.e., pro-inflammatory switch), 3) enhancing glutathione enzymes that prevent oxidative damage and oxidative stress and, 4) increases DNA methylation to control cell proliferation (Gegotek et al, 2019 Cells; Pucci et al., 2013).

Equol is commercially available from LC labs, Woburn, Mass., USA.

Resveratrol

In certain embodiments, the described compositions may include resveratrol, which is an example of a polyphenolic compound similar to equol. A comparison of the chemical structures for 17β-estradiol, resveratrol and equol are shown in FIG. 8.

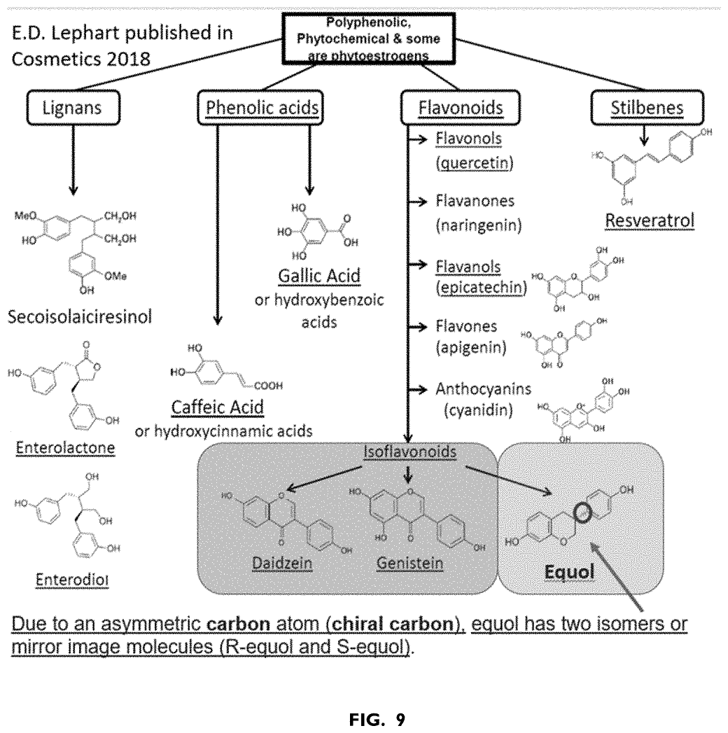

Resveratrol has been reported to protect mammals against radiation exposure (Dobrzynska et al., 2016), suggesting that this polyphenolic compound may effectively countermeasure the dangers posed by radiation exposure. The chemical structures of equol and resveratrol are shown in FIG. 8. The general polyphenolic classification of phytochemical molecules and the characteristics of the isoflavonoids, specifically is displayed in FIG. 9.

In certain embodiments, more than one polyphenolic compound may be included in the mixture. For example, in certain embodiments, equol and resveratrol may be combined in the composition. As equol has been previously shown to enhance the actions of resveratrol, equol may have a further positive impact on another polyphenolic molecules and the potential use of equol, equol analogs and other polyphenolic molecules, like resveratrol, is contemplated in the composition described herein. In certain embodiments, equol may be combined to generate an effective treatment for symptoms or conditions due to a radiation exposure along with other various disorders like certain types of cancer, dermal, muscle, inflammatory, neurological, heart, metabolic and endocrine diseases or disorders.

In the described compositions, polyphenolic compounds may form anywhere from 0.01 to 99% by weight/weight of the composition. For example, preferably the polyphenolic compound(s) forms at least 1% w/w of the composition; more preferably, at least 5% w/w of the composition; more preferably; at least 10% w/w of the composition; more preferably, at least 15% w/w of the composition; more preferably, at least 20% w/w of the composition; more preferably at least 25% w/w of the composition; more preferably, at least 30% w/w of the composition; more preferably, at least 30% w/w of the composition; more preferably, at least 35% w/w of the composition; more preferably, at least 40% w/w of the composition; more preferably, at least 45% w/w of the composition; more preferably at least 50% or more w/w of the composition. Any dosage amount or percentages in-between the above amounts are also contemplated.

Astaxanthin

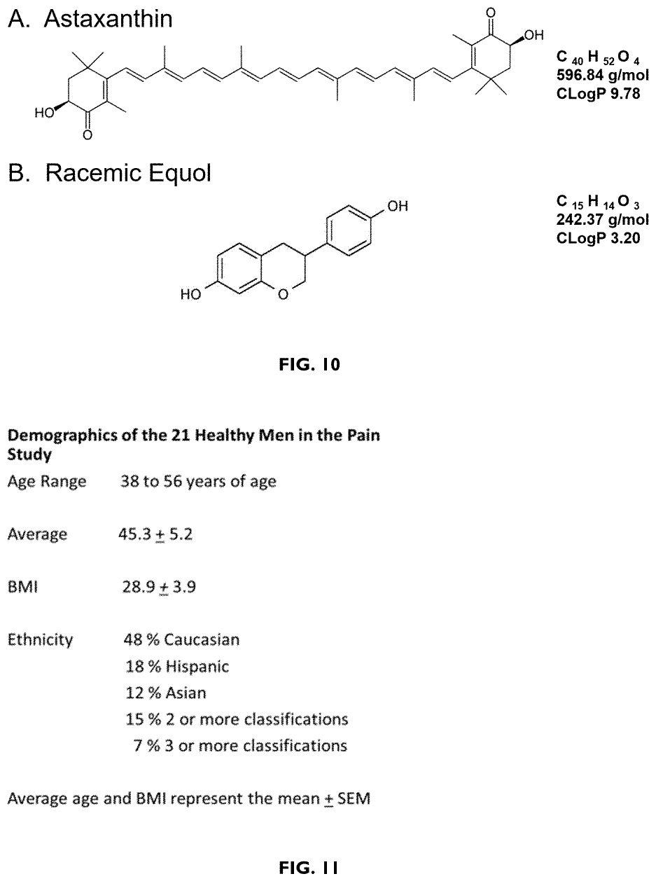

In one embodiment, the described composition may include yet another polyphenolic compound, such as astaxanthin. Astaxanthin is a phytochemical compound that is a known powerful antioxidant and anti-inflammatory agent. The chemical structures and chemical properties of astaxanthin and equol are displayed in FIG. 10. In a recent study, equol was shown to significantly alter 39 human skin biomarkers; astaxanthin influenced 6 skin genes (compared to equol). The results revealed significant effects of equol and astaxanthin on the antioxidants, growth factors, extracellular integrity and extracellular breakdown, and the inflammatory biomarkers (Lephart, 2019).

In summary, equol, resveratrol and astaxanthin are powerful antioxidant, anti-inflammatory agents. Equol is a powerful inhibitor of oxidative stress and has other properties of relevance to cellular, tissue, organ and biological function where approximately 50% of equol circulates in the free or unbound form in plasma (Lephart, 2016); selectively binds estrogen receptor beta that has been reported to be protective against prostate and breast cancers and neuroprotective against mitochondrial dysfunction, stroke (cerebral focal ischemia), hypoxia, memory, cognitive function and neurodegenerative diseases (Lephart, 2016).

It has been reported that equol acts as a potent radio-sensitizer in estrogen-positive and -negative human breast cancer cell lines that increase cell death following irradiation (Taghizadeh et al., 2015). Thus, the preventive actions of pretreatment with equol would protect normal cells and tissues and point to the need to have both pre- and post-treatment regiments of equol for radiation exposure.

In the composition, equol (racemic or non-racemic) may form from about 0.1% to 10% for topical applications, and 1 mg to 15 mg for oral applications.

In the composition, resveratrol may form from about 0.1% to 10% for topical applications, and 1 mg to 500 mg for oral applications.

In the composition, astaxanthin may form from about 0.1% to 10% for topical applications, and 1 mg to 12 mg for oral applications.

The phytochemicals such as equol, resveratrol, astaxanthin etc. are commercially available from LC Labs, Woburn, Mass., USA.

C. Other Compounds

Also, the described compositions may include at least one other active ingredient selected from the group consisting of collagen, vitamin C, vitamin E, zinc, grape seed extract, niacinamide, hydroxy acids, hyaluronic acid, and coenzyme Q10.

Other natural active ingredients such as collagen, vitamin C, vitamin E, zinc, grape seed extract, niacinamide, hydroxy acids, hyaluronic acid, or coenzyme Q10 are known to have important effects on human skin, muscle, nerve, and inflammation (Beyer, 1990; Overvard et al., 1999; Inui et al., 2008; Zhang et al., 2012; Hargreaves, 2014; Lephart, 2015; Lephart, 2016; Lephart, 2017; Tang & Yang, 2018; Draelos, 2019; Lephart, 2019; Zaid & Ramahi, 2019; Naftolin and Lephart, 2021 (in press); Odah et al., 2020; Woodby et al., 2020), however, their use in combination with CBD and/or a polyphenolic compound(s) have not been described for the prevention or treatment of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject.

Collagen

In certain further embodiments, the described compositions may include collagen.

Collagen is the most abundant protein in the human body where it is responsible for structure, stability, and strength especially within the dermal layers (Ricard-Blum, 2011; Kohl et al., 2011; Farage et al., 2017; Lephart, 2018a). With regard to aging, the deposition of collagen (and elastin) decreases with chronological-aging (with the passage of time) and particularly with photo-aging (exposure to the sun) (Kohl et al., 2011; Farage et al., 2017; Lephart, 2016; Lephart 2018a). In addition, it can be broken down by hydrolyzing proteins such as matrix metalloproteinases (MMPs), which results in dermal damage and undesirable skin wrinkles (Kohl et al., 2011; Farage et al., 2017; Lephart 2018a).

Collagen provides support to various tissues such as tendons, ligaments, skin, teeth, and many other connective tissue structures.

So far, 28 types of collagen have been identified and described that can be grouped into eight families depending on its structure (Kohl et al., 2011; Ricard-Blum, 2011; Farage et al., 2017; Rodriguez et al., 2017; Lephart 2018a). All collagen proteins have a structure based on three helical polypeptide chains with glycine (Gly) occurring every three amino acid residues while proline (Pro) and hydroxyproline (Hyp) make up about ⅙ of the total sequence of collagen (Clark et al., 1935; Rodriguez et al, 2017). The sequence often follows the pattern Gly-Pro-X or Gly-X-Hyp where X may be any of various other amino acid residues (Liu et al., 2015). A typical collagen triple-helix structure can have a diameter of 10 to 500 nm, an approximate molecular weight of 285 kDa, and is composed of 1400 amino acids (Rodriguez et al., 2017). Collagen is a viscoelastic material with high tensile strength with low immunogenicity where it can be ingested or injected into a foreign body and can be further modified to eliminate any immune response by heat or chemical treatment (Farage et al., 2017; Rodriguez et al., 2017). Any type of collagen can be used in the described composition, however, marine sources of collagen, which have advantages over animal sources due to their greater absorption from their low molecular weight, and negligible biological contaminants such as toxins and low inflammatory effects are more feasible for commercial use (Vollmer et al., 2018).

Collagen can be obtained from animal and vegetable sources with the most common coming from bovine, porcine, human, and marine organisms such as fish scale and fish skin (Rodriguez et al., 2017). It is known that collagen hydrolysate has several positive biological properties such as antioxidant, antihypertensive, and lipid-lowering activities as well as the established reparative actions in damaged skin (Khol et al., 2011; Farage et al., 2017; Rodriguez et al., 2017; Lephart, 2018a). Furthermore, collagen has a dual action in the skin where it first provides the building block components for collagen (and elastin) and, secondly, it binds receptors in fibroblasts located in the dermal layers to stimulate the synthesis of collagen and elastin as well as hyaluronic acid (Khol et al., 2011; Farage et al., 2017; Rodriguez et al., 2017; Lephart, 2018a).

In the described compositions, collagen may form from about 0.5 gram % to about 5 grams % w/w of the composition.

Collagen is commercially available from Making Cosmetics Inc., Redmond, Wash., USA and BulkSupplements.com, Las Vegas, Nev., USA.

Coenzyme Q10

In certain further embodiments, the described compositions may include coenzyme Q.

Coenzyme Q10 is an endogenous lipid soluble antioxidant present in all membranes, is the cofactor for three mitochondrial enzymes (complexes I, II, and III), and is known to reduce mitochondrial oxidative damage (Vollmer et al., 2018). The mechanism of action of coenzyme Q10 as an antioxidant has been shown to: (a) reduce the production of free radicals, (b) be involved in the regeneration of vitamin E, (c) reduce keratinocyte DNA damage, (d) reduce UVA-induced MMP production in fibroblasts, (e) enhance collagen and elastin expression, inhibit IL-1α, IL-6 production, and melanin synthesis, and (f) inhibit MMPs and regulate the sulfide oxidation pathway (Muta-Takada et al., 2009; Knott et al., 2015; Hernandez-Camacho et al., 2018; Vollmer et al., 2018).

In the composition, coenzyme Q may form from about 0.1% to about 3% w/w of the composition (topical application); from about 50 mg to about 1200 mg may be used for oral applications.

Coenzyme Q is commercially available from Making Cosmetics Inc., Redmond, Wash., USA or BulkSupplements LLC, Las Vegas, Nev., USA.

Vitamin C

In yet other embodiments, the described compositions may include vitamin C.

Vitamin C can be used in many forms such as magnesium ascorbyl phosphate, L-ascorbic acid, sodium ascorbyl phosphate, 3-glyceryl ascorbate, L-ascorbyl palmitate, tetrahexyldecyl ascorbate, 3-O-ethyl ascorbate and ascorbyl 2-glucoside, among many others. Along with enhancers like ferulic acid and vitamin E that can stabilize and increase vitamin C's effects in many health applications, especially in topical treatments.

Vitamin C's skin health promoting effects include: a) an antioxidant that neutralizes free radicals to help protect the skin from precancerous dermal changes caused by UV exposure, b) an accelerator for the production of collagen and elastin, the basic proteins in the extracellular matrix; in fact, TGBβ and vitamin C act in synergy with respect to collagen deposition (Telang, 2013; Piesma et al., 2017; Pullar et al., 2017; Ravetti et al., 2019), c) an inhibitor of melanin production, which helps prevent dark spots from forming and d) a superior brightening agent that increases the skin radiance (Draelos, 2019; Ravetti et al., 2019; Zaid & Ramahi, 2019; Vitamin C and Skin, 2020; Woodby et al., 2020). Finally, the use of enhancers of vitamin C can be used. For example, we have determined that a combination of 0.5% of ferulic acid (a potent antioxidant of plant origin) with 15% vitamin C and 1% vitamin E can increase the efficacy of vitamin C by 8-old to reduce acute and chronic photo-skin damage and prevent skin cancer.

In the composition, vitamin C may form from about 3% to about 25% w/w of the composition (topical applications); from 20 mg to 150 mg or oral applications.

Vitamin C is commercially available from Making Cosmetics Inc., Redmond, Wash., USA or BulkSupplements.com, Las Vegas, Nev., USA.

Zinc

In yet further embodiments, the described compositions may include zinc.

Zinc is a natural trace mineral and plays an important role in three skin functions such as morphogenesis, repair, and maintenance that provide protection and defense via the proteins and enzymes that are involved in these processes (Prasad, 2017; Vollmer et al., 2018). Approximately 6% of the total concentration of the body's zinc is located in skin, which is present at levels five-fold higher in the epidermis when compared to the dermis (Michaelsson et al., 1980; Prasad, 2017; Vollmer et al., 2018). Zinc is known to stabilize cell membranes, act as an essential cofactor for several metalloenzymes (MMPs), be involved with superoxide dismutase, metallothionein, DNA, and RNA polymerases, and participate in basal cell mitosis and differentiation (Prasad, 2017; Vomer et al., 2018). Zinc is also needed for the body's defensive (immune) system to properly work. It plays a role in cell division, cell growth, wound healing, and the breakdown of carbohydrates (Prasad, 2018; Vollmer et al., 2018).

In the composition, zinc may form from about 3% to about 10% w/w of the composition (topical applications); from about 2 mg to about 11 mg for oral applications.

Zinc is commercially available from Making Cosmetics Inc., Redmond, Wash., USA or BulkSupplements.com Las Vegas, Nev., USA.

Grape Seed Extract

Yet in further embodiments, the described compositions may include grape seed extract.

Grape seed extract (GSE) is a great source of antioxidants, vitamin C and vitamin E which protects the skin from harmful irritants such as UV light, pollution, sun damage, smoke, and free radicals (Bergfeld et al., 2012; Costa et al., 2015; Devi & Singh, 2017; Gupta et al., 2020). GSE on its own has many skin anti-aging properties to help fight the battle against the formation of fine lines and wrinkles (Costa et al., 2015; Devi & Singh, 2017; Odah et al., 2020).

GSE has been reviewed by the cosmetic ingredient review board (via an expert panel of scientists) in Washington, D.C., USA to be effective and safe for use in cosmetics (Bergfeld et al., 2012). Additionally, grape seed extract provides benefits against many diseases i.e. inflammation, cardiovascular disease, hypertension, diabetes, cancer, peptic ulcer, and microbial infections, etc. (Gupta et al., 2020).

In the composition, GSE may form from about 2% to about 5% w/w of the composition (for topical applications); and from about 2 mg to 200 mg for oral applications.

GSE is commercially available from Making Cosmetics Inc., Redmond, Wash., USA and BulkSupplements.com, Las Vegas, Nev., USA

Nicotinamide

In certain further embodiments, the described compositions may include nicotinamide.

Nicotinamide, also known as niacinamide, is the amide form of vitamin B3. It is a precursor of essential coenzymes for numerous reactions in the body including adenosine triphosphate (ATP) production.

Nicotinic acid, also known as niacin, is converted into nicotinamide in the body. The use of topical nicotinamide in the treatment of acne vulgaris; melasma; atopic dermatitis; rosacea; and oral nicotinamide in preventing nonmelanoma skin cancer has shown positive effects and has been previously reported.

In the composition, nicotinamide may form from about 0.3% to about 5% w/w of the composition (for topical applications); and from about 1 mg to about 500 mg for oral applications.

Nicotinamide is commercially available from BulkSupplements.com, Las Vegas, Nev., USA or LC Labs, Woburn, Mass., USA.

Hydroxy Acids

In yet further embodiments, the described compositions may include a hydroxyl acid.

Hydroxy acids or alpha-hydroxy acids (AHAs) include glycolic acid (GA), citric acid (CA), malic acid (MA), tartaric acid (TA), and lactic acid (LA), all of which are naturally-occurring organic acids present in many foods and milk sugars (Zaid & Ramahi, 2019; Tang & Yang, 2018). For example, AHAs are found throughout nature in sugarcane (glycolic acid), sour milk (lactic acid), and fruits (citric acid and malic acid) (Tang & Yang, 2018). AHAs are often used as ingredients for skin cleaning products. AHAs at low concentrations may be beneficial to the skin because of epigenetic modifications of inflammasome complex. AHAs have dual effects on the skin, beneficial at low levels and inflammatory effects at high concentrations (Tang & Yang, 2018).

AHAs may be present in the composition at from 1% to about 10% w/w of the composition (for topical applications).

AHAs may be commercially available from Making Cosmetics Inc., Redmond, Wash., USA.

Hyaluronic Acid

In yet another embodiment, hyaluronic acid (HA) may be included in the described compositions.

HA is a molecule present in many strains of bacteria and is ubiquitous in all vertebrates, where it is particularly abundant in the embryonic tissues and in the extracellular matrix (ECM) of adult soft connective tissues (Abatangelo et al., 2020).

HA is synthesized within cells and is widely present in the ECM of the skin, the largest organ of the human body, and its presence is fundamental for the rheological, hygroscopic and viscoelastic properties of the tissue (Litwiniuk et al., 2016; Gupta et al., 2019; Abatangelo et al., 2020). Although it has been shown that this polysaccharide is also associated with repair, HA has been demonstrated to play a crucial role by influencing inflammatory, proliferative, or re-modeling phases of skin healing process (Litwiniuk et al., 2016; Gupta et al., 2019; Zaid & Ramahi, 2019; Abatangelo et al., 2020).

The available data suggest that HA homeostasis exhibits a distinct profile in intrinsic skin aging, which is totally different of that in extrinsic skin aging. HA has been shown to play a multifaceted role in regulating various biological processes such as skin repairmen, wound healing, tissue regeneration, anti-inflammatory, and immunomodulation. Owing to its remarkable biomedical and tissue regeneration potential, HA has been numerously employed as one of the imperative components of the cosmetic and nutricosmetic products (Bukhari et al., 2018). Additionally, there are several other health applications of HA that include vascular tissue & peripheral nerve repair, osteoarthritis, and cancer therapy (Abatangelo et al., 2020).

HA, sodium hyaluronate and its derivatives for cosmetic formulations are typically derived from vegan sources as hyaluronic acid powder that can be mixed with different combination(s) of ingredients for topical applications. Additionally, HA comes in different molecular weights that determine the action and penetration into the skin where higher molecular weights of HA do not absorb into the dermal layers well, whereas, lower molecular weights of HA penetrate into the deeper skin layers (Essendoubi et al., 2016). However, high molecular weight HA can passively penetrate the skin using nanoparticles as reported by Tokudome et al., in 2018.

In the composition, HA can form from about 0.1% to about 5% of the composition for topical applications); and from about 50 mg to about 300 mg for oral applications.

HA may be commercially available from Making Cosmetics Inc., Redmond, Wash., USA and BulkSupplements.com, Las Vegas, Nev., USA.

II. Methods

In one embodiment, described herein is a method for the prevention and/or treatment of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject, comprising administering to the subject a composition comprising: (i) one or more polyphenolic compound, its isomer or analog; and (ii) one or more other active ingredient selected from the group consisting of collagen, vitamin C, vitamin E, Zinc, grape seed extract, niacinamide, hydroxy acids, hyaluronic acid, and coenzyme Q10.

In another embodiment, described herein is a method for the prevention and/or treatment of at least one condition or disorder of skin and accessory structures of the skin and/or for improving the health status of a subject, comprising administering to the subject a composition comprising (a) cannabidiol (CBD); and (b) at least one of: (i) one or more polyphenolic compound, an isomer, analog, or a derivative thereof, and (ii) one or more other active ingredient selected from the group consisting of collagen, vitamin C, vitamin E, zinc, grape seed extract, niacinamide, hydroxy acids, hyaluronic acid, and coenzyme Q10.

Various applications include cosmetic and dermatology applications in the public domain and, medical/dental, veterinarian, environmental work conditions.

Exemplary conditions or disorders of the skin and the accessory structures of the skin include skin rash (i.e., nearly any change in the skin's appearance can be called a rash—most rashes are from simple skin irritation; others result from medical conditions); dermatitis (i.e., an inflammation of the skin), including atopic dermatitis (a type of eczema); eczema (i.e., skin inflammation (dermatitis) causing an itchy rash, most often, it's due to an overactive immune system); psoriasis (i.e., an autoimmune condition that can cause a variety of skin rashes); dandruff (i.e., a scaly condition of the scalp may be caused by seborrheic dermatitis, psoriasis, or eczema); acne (i.e., the most common skin condition, acne affects over 85% of people at some time in life); cellulitis (i.e., inflammation of the dermis and subcutaneous tissues, usually due to an infection; a red, warm, often painful skin rash generally results); skin abscess (e.g., boil or furuncle; a localized skin infection creates a collection of pus under the skin); rosacea (i.e., a chronic skin condition causing a red rash on the face); warts; melanoma (i.e., type of skin cancer); basal cell carcinoma (type of skin cancer); seborrheic keratosis (i.e., a benign, often itchy growth that appears like a “stuck-on” wart); actinic keratosis (i.e., a crusty or scaly bump that forms on sun-exposed skin); squamous cell carcinoma (i.e., a common form of skin cancer, squamous cell carcinoma may begin as an ulcer that won't heal, or an abnormal growth); herpes (the herpes viruses HSV-1 and HSV-2 can cause periodic blisters or skin irritation around the lips or the genitals); hives (i.e., raised, red, itchy patches on the skin that arise suddenly, which usually result from an allergic reaction); tinea versicolor (i.e., a benign fungal skin infection creates pale areas of low pigmentation on the skin); viral exantham (i.e., many viral infections can cause a red rash affecting large areas of the skin); shingles (herpes zoster); scrabies (i.e., tiny mites that burrow into the skin cause scabies); ringworm (i.e., fungal skin infection (also called tinea); aging skin; wound healing; dry skin; damaged skin barrier; and scalp hair loss. In addition, dermal health—for example—covering skin anti-aging includes chronological (intrinsic) and photo- (extrinsic) aging, augmentation of collagen, elastin, wound healing, facial color and tone, smoothness, skin integrity (barrier repair), hyaluronan and other disaccharides, scar healing, antioxidant factors/protection, enhancement of finger nail health, and defend against matrix metalloproteinases, elastase, fibroblast collapse, inflammatory biomarkers, psoriasis, atopic dermatitis, acne, skin dryness, skin itch, scalp hair loss and skin thinning.

Exemplary conditions of the musculoskeletal system include, e.g., pain, such as muscle and joint pain, muscle cramps, movement disorders, osteoporosis, and neuromuscular conditions.

Exemplary conditions of the cardiovascular system include, e.g., hypertension, heart disease, stroke, tissue hypoxia, blood vessel integrity.

Conditions of the inflammatory system relate to conditions where the immune system attacks the body's own tissues, resulting in inflammation. Exemplary inflammatory conditions include, e.g., rheumatoid arthritis. The described compositions can be used to decrease the oxidative stress, inhibit NFKappa B, protect cells against free radicals, and enhance Nrf2 defense mechanisms (antioxidant production), thereby aid in protecting against or treating inflammation.

Exemplary conditions of the neurological system that may be treated with the described composition include memory loss, decreased cognitive function, neurodegenerative disorders, e.g., Alzheimer's disease, Parkinson's disease, progressive supranuclear palsy, progressive white matter brain lesions with aging or trauma, cranial blood vessel integrity and other related disorders, fibromyalgia.

Exemplary cancers that may be treated with the described composition include skin cancer and other tissue and organ cancers, NA mutations, radiation burns, radiation sickness, and premature aging.

Exemplary endocrine/metabolic health conditions that may be treated with the described composition include weight gain or loss (e.g., weight control), hypercholesterolemia, pituitary, adrenal, pancreatic (diabetes and insulin production) and thyroid disorders.

Administration

The described composition may be administered via oral administration, topical administration, trans-dermal patches, sprays, lotions, injections (intra-muscular) and intra-venous administration. Administration of topical CBD in combination with other polyphenolic compounds (e.g., equol, resveratrol, genistein, daidzein, lignans, phenolic acids, caffeic acids, flavonoids and other isoflavonoids, astaxanthin, etc.) may be represented by embodiments of skin formulations that could include dosing of CDB at 1 mg/ml to 20 mg/ml, with preferred dosing at 5 mg/ml to 15 mg/ml, and equol at 0.1% to 10% with preferred dosing at 0.1 to 3% (see Table 2 for embodiments for skin formulations). Also, topical skin formulations could include resveratrol at 0.1% to 10% and astaxanthin at 0.1% to 10%.

In addition to CDB and polyphenol compounds, like equol or other natural ingredients may embody skin topical formulation(s) that include: vitamin C at 3% to 25% with preferred dosing at 8% to 20%; vitamin E at 0.3% to 3% with preferred dosing at 0.8% to 2%; ferulic acid at 0.1% to 3% with preferred dosing at 0.3% to 1%; grape seed extract at 2% to 15% with preferred dosing at 3% to 8%; niacinamide at 0.1% to 5% with preferred dosing at 1% to 3.5%; hyaluronic acid at 0.1% to 5% with preferred dosing at 1% to 3.5% and coenzyme Q10 at 0.1% to 3% with preferred dosing at 0.3% to 1% (Table 2). Notably, the dosage may be higher to treat conditions like wound healing, acne, etc. For example, CBD at 10 to 20 mg/ml; equol at 3 to 10%; vitamin C at 15 to 25%; vitamin E at 2 to 3%; ferulic acid at 0.5 to 3%; grape see extract at 3 to 15%; niacinamide at 0.5 to 5%; hyaluronic acid at 0.5 to 5% and coenzyme Q10 at 0.5 to 3%.

Exemplary formulations based on the descried embodiments are provided in Table 2 below.

TABLE 2
Examples of Skin Topical Formulation(s) (STF) and description(s)
of preferred embodiment of each formula.

| | |
|---|---|
| Ingredient | Dose |

Skin Topical Formulation (STF 1):

| | |
|---|---|
| Cannabidiol (CDB) | (1 mg/ml to 20 mg/ml; preferred |
| | 5 mg/ml to 15 mg/ml) |
| Equol (racemic or non-racemic) | (0.1% to 10%; preferred 0.1% to 3%) |
| Vitamin C | (3% to 25%; preferred 8% to 20%) |
| Vitamin E | (0.3% to 3%; preferred 0.8% to 2%) |
| Ferulic Acid | (0.1% to 2%; preferred 0.3% to 1%) |
| Grape Seed Extract | (2% to 15%; preferred 3 to 8%) |
| Niacinamide | (0.3% to 5%; preferred 1% to 3.5%) |
| Hyaluronic Acid | (0.1% to 5%; preferred 1% to 3%) |
| Coenzyme Q10 | (0.1% to 3%; preferred 0.1 to 1%) |

Skin Topical Formulation (STF2):

| | |
|---|---|
| Cannabidiol (CDB) | (1 mg/ml to 20 mg/ml; preferred |
| | 5 mg/ml to 15 mg/ml) |
| Equol (racemic or non-racemic) | (0.1% to 10%; preferred 0.1% to 3%) |

Skin Topical Formulation (STF3):

| | |
|---|---|
| Cannabidiol (CDB) | (1 mg/ml to 20 mg/ml; preferred |
| | 5 mg/ml to 15 mg/ml) |
| Vitamin C | (3% to 25%; preferred 8% to 20%) |
| Vitamin E | (0.3% to 3%; preferred 0.8% to 2%) |
| Ferulic Acid | (0.1% to 2%; preferred 0.3% to 1%) |
| Grape Seed Extract | (2% to 15%; preferred 3 to 8%) |
| Niacinamide | (0.3% to 5%; preferred 1% to 3.5%) |
| Hyaluronic Acid | (0.1% to 5%; preferred 1% to 3%) |
| Coenzyme Q10 | (0.1% to 3%; preferred 0.1 to 1%) |

Skin Topical Formulation (STF4):

| | |
|---|---|
| Equol (racemic or non-racemic) | (0.1% to 10%; preferred 0.1% to 3%) |
| Vitamin C | (3% to 25%; preferred 8% to 20%) |
| Vitamin E | (0.3% to 3%; preferred 0.8% to 2%) |
| Ferulic Acid | (0.1% to 2%; preferred 0.3% to 1%) |
| Grape Seed Extract | (2% to 15%; preferred 3 to 8%) |
| Niacinamide | (0.3% to 5%; preferred 1% to 3.5%) |
| Hyaluronic Acid | (0.1% to 5%; preferred 1% to 3%) |
| Coenzyme Q10 | (0.1% to 3%; preferred 0.1 to 1%) |

Skin Topical Formulation (STF5):

| | |
|---|---|
| Equol (racemic or non-racemic) | (0.1% to 10%; preferred 0.1% to 3%) |
| Vitamin C | (3% to 25%; preferred 8% to 20%) |
| Vitamin E | (0.3% to 3%; preferred 0.8% to 2%) |
| Ferulic Acid | (0.1% to 2%; preferred 0.3% to 1%) |
| Grape Seed Extract | (2% to 15%; preferred 3 to 8%) |
| Niacinamide | (0.3% to 5%; preferred 1% to 3.5%) |
| Hyaluronic Acid | (0.1% to 5%; preferred 1% to 3%) |

Skin Topical Formulation (STF6):

| | |
|---|---|
| Equol (racemic or non-racemic) | (0.1% to 10%; preferred 0.1% to 3%) |
| Vitamin C | (3% to 25%; preferred 8% to 20%) |
| Vitamin E | (0.3% to 3%; preferred 0.8% to 2%) |
| Ferulic Acid | (0.1% to 2%; preferred 0.3% to 1%) |
| Grape Seed Extract | (2% to 15%; preferred 3 to 8%) |
| Hyaluronic Acid | (0.1% to 5%; preferred 1% to 3%) |

Skin Topical Formulation (STF7):

| | |
|---|---|
| Equol (racemic or non-racemic) | (0.1% to 10%; preferred 0.1% to 3%) |
| Vitamin C | (3% to 25%; preferred 8% to 20%) |
| Vitamin E | (0.3% to 3%; preferred 0.8% to 2%) |
| Ferulic Acid | (0.1% to 2%; preferred 0.3% to 1%) |
| Hyaluronic Acid | (0.1% to 5%; preferred 1% to 3%) |

Skin Topical Formulation (STF8):

| | |
|---|---|
| Equol (racemic or non-racemic) | (0.1% to 10%; preferred 0.1% to 3%) |
| Vitamin C | (3% to 25%; preferred 8% to 20%) |
| Vitamin E | (0.3% to 3%; preferred 0.8% to 2%) |
| Ferulic Acid | (0.1% to 2%; preferred 0.3% to 1%) |

Skin Topical Formulation (STF9):

| | |
|---|---|
| Equol (racemic or non-racemic) | (0.1% to 10%; preferred 0.1% to 3%) |
| Vitamin C | (3% to 25%; preferred 8% to 20%) |
| Vitamin E | (0.3% to 3%; preferred 0.8% to 2%) |

Skin Topical Formulation (STF 10):

| | |
|---|---|
| Equol (racemic or non-racemic) | (0.1% to 10%; preferred 0.1% to 3%) |
| Vitamin C | (3% to 25%; preferred 8% to 20%) |

The CBD and polyphenolic compounds include isomers, analogs and derivatives of the original molecule described herein. Skin and tissue penetrating enhancers along with methods to absorb the CBD plus polyphenolic compound treatments via oral or other methods of delivery for health applications and could include cosmetic, pharmaceutical, drug, food components and additives such as transcutol, organic and synthetic compounds, and micro-encapsulation, etc.

Administration of oral CBD in combination with other polyphenolic compounds (like equol, resveratrol, genistein, daidzein, lignans, phenolic acids, caffeic acids, flavonoids and other isoflavonoids, astaxanthin, etc.) may be represented by embodiments of formulations that may be orally administered include dosing of CDB at 10 mg to 800 mg with preferred dosing at 20 mg to 200 mg; polyphenol compounds like equol at 1 mg to 15 mg with preferred dosing at 2 to 6 mg (Table 3). Also orally administered formulation include dosing of resveratrol at 1 mg to 500 mg and astaxanthin at 1 mg to 12 mg. In addition to CDB and polyphenol compounds, like equol other natural ingredients may embody oral formulation(s) that include: collagen at 0.5 grams to 5 grams with preferred dosing at 2 to 3 grams; vitamin C at 20 to 500 mg with preferred dosing at 75 to 250 mg; vitamin E at 2 mg to 120 mg with preferred dosing at 10 mg to 55 mg; hyaluronic acid at 50 mg to 300 mg with preferred dosing at 100 to 200 mg; and coenzyme Q10 at 50 mg to 1,200 mg with preferred dosing at 100 to 300 mg (Table 3). Notably, the dosage may be higher to treat conditions like joint/muscle pain and/or cramping, and for brain health conditions etc. For example, CDB at 30 to 800 mg; equol at 6 to 15 mg; collagen at 2 to 5 grams; vitamin C at 50 to 150 mg; vitamin E at 10 to 20 mg; hyaluronic acid at 100 to 300 mg and coenzyme Q10 at 300 mg to 1200 mg.

TABLE 3
Examples of Oral Formulation(s) (OF) and description(s)
of preferred embodiment of each formula.

| | |
|---|---|
| Ingredient | Dose |

Oral Formulation (OF 1):

| | |
|---|---|
| Cannabidiol (CDB) | (10 mg to 800 mg; preferred 20 mg to 150 mg) |
| Equol (racemic or | (1 mg to 15 mg; preferred 2 to 6 mg or 5 to 10 mg) |
| non-racemic) | |
| Collagen | (0.5 to 5 grams; preferred 2 to 3 grams) |
| Vitamin C | (20 mg to 150 mg; preferred 75 mg to 90 mg) |
| Vitamin E | (2 mg to 20 mg; preferred 5 mg to 10 mg) |
| Hyaluronic acid | (50 mg to 300 mg; preferred 100 mg to 200 mg) |
| Coenzyme Q10 | (50 mg to 1200 mg; preferred 100 mg to 300 mg) |

Oral Formulation (OF 2):

| | |
|---|---|
| Cannabidiol (CDB) | (10 mg to 800 mg; preferred 20 mg to 150 mg) |
| Equol (racemic or | (1 mg to 15 mg; preferred 2 to 6 mg or 5 to 10 mg) |
| non-racemic) | |

Oral Formulation (OF 3):

| | |
|---|---|
| Cannabidiol (CDB) | (10 mg to 800 mg; preferred 20 mg to 150 mg) |
| Collagen | (0.5 to 5 grams; preferred 2 to 3 grams) |
| Vitamin C | (20 mg to 150 mg; preferred 75 mg to 90 mg) |
| Vitamin E | (2 mg to 20 mg; preferred 5 mg to 10 mg) |
| Hyaluronic acid | (50 mg to 300 mg; preferred 100 mg to 200 mg) |
| Coenzyme Q10 | (50 mg to 1200 mg; preferred 100 mg to 300 mg) |

Oral Formulation (OF 4):

| | |
|---|---|
| Equol (racemic or | (1 mg to 15 mg; preferred 2 to 6 mg or 5 to 10 mg) |
| non-racemic) | |
| Collagen | (0.5 to 5 grams; preferred 2 to 3 grams) |
| Vitamin C | (20 mg to 150 mg; preferred 75 mg to 90 mg) |
| Vitamin E | (2 mg to 20 mg; preferred 5 mg to 10 mg) |
| Hyaluronic acid | (50 mg to 300 mg; preferred 100 mg to 200 mg) |
| Coenzyme Q10 | (50 mg to 1200 mg; preferred 100 mg to 300 mg) |

Oral Formulation (OF 5):

| | |
|---|---|
| Equol (racemic or | (1 mg to 15 mg; preferred 2 to 6 mg or 5 to 10 mg) |
| non-racemic) | |
| Collagen | (0.5 to 5 grams; preferred 2 to 3 grams) |
| Vitamin C | (20 mg to 150 mg; preferred 75 mg to 90 mg) |
| Vitamin E | (2 mg to 20 mg; preferred 5 mg to 10 mg) |
| Hyaluronic acid | (50 mg to 300 mg; preferred 100 mg to 200 mg) |

Oral Formulation (OF 6):

| | |
|---|---|
| Equol (racemic or | (1 mg to 15 mg; preferred 2 to 6 mg or 5 to 10 mg) |
| non-racemic) | |
| Collagen | (0.5 to 5 grams; preferred 2 to 3 grams) |
| Vitamin C | (20 mg to 150 mg; preferred 75 mg to 90 mg) |
| Vitamin E | (2 mg to 20 mg; preferred 5 mg to 10 mg) |

Oral Formulation (OF 7):

| | |
|---|---|
| Equol (racemic or | (1 mg to 15 mg; preferred 2 to 6 mg or 5 to 10 mg) |
| non-racemic) | |
| Collagen | (0.5 to 5 grams; preferred 2 to 3 grams) |
| Vitamin E | (2 mg to 20 mg; preferred 5 mg to 10 mg) |
| Hyaluronic acid | (50 mg to 300 mg; preferred 100 mg to 200 mg) |

Oral Formulation (OF 8):

| | |
|---|---|
| Equol (racemic or | (1 mg to 15 mg; preferred 2 to 6 mg or 5 to 10 mg) |
| non-racemic) | |
| Collagen | (0.5 to 5 grams; preferred 2 to 3 grams) |
| Coenzyme Q10 | (50 mg to 1200 mg; preferred 100 mg to 300 mg) |

Oral Formulation (OF 9):

| | |
|---|---|
| Equol (racemic or | (1 mg to 15 mg; preferred 2 to 6 mg or 5 to 10 mg) |
| non-racemic) | |
| Collagen | (0.5 to 5 grams; preferred 2 to 3 grams) |
| Hyaluronic acid | (50 mg to 300 mg; preferred 100 mg to 200 mg) |

Oral Formulation (OF 10):

| | |
|---|---|
| Equol (racemic or | (1 mg to 15 mg; preferred 2 to 6 mg or 5 to 10 mg) |
| non-racemic) | |
| Collagen | (0.5 to 5 grams; preferred 2 to 3 grams) |
| Vitamin E | (2 mg to 20 mg; preferred 5 mg to 10 mg) |

Oral Formulation (OF 11):

| | |
|---|---|
| Equol (racemic or | (1 mg to 15 mg; preferred 2 to 6 mg or 5 to 10 mg) |
| non-racemic) | |
| Collagen | (0.5 to 5 grams; preferred 2 to 3 grams) |
| Coenzyme | Q10 (50 mg to 1200 mg; preferred 100 mg to |
| | 300 mg)) |

Oral Formulation (OF 12):

| | |
|---|---|
| Equol (racemic or | (1 mg to 15 mg; preferred 2 to 6 mg or 5 to 10 mg) |
| non-racemic) | |
| Collagen | (0.5 to 5 grams; preferred 2 to 3 grams) |

Equol (racemic or non-racemic) preferred dose (low: 2 to 6 mg to high: 6 to 15 mg) dependent upon age and health condition.

### EXAMPLES

### Example 1: Treatment with CBD and Equol for Muscle Pain

Methods:

A total of 21 healthy adult men were studied using the short-form McGill pain questionnaire (Melzack, 1987). The demographics for the 21 healthy adult men in this study are shown in FIG. 11. A total of 5 healthy men were tested for each body region (neck/shoulder, arm, upper back, lower back or leg/knee), where the pain rating was determined before treatment and then post-treatment, therefore, n=5 for each body region.

The typical body regions where pain is experienced includes: 22% report neck pain, 21% report shoulder pain, 13% report upper back pain, 25% report lower back pain, 7% report arm pain and 12% report leg pain.

The subjects were treated with a composition comprising the combination of CBD (at 8 mg) and 0.3% of racemic equol (per dose) administered in a cream/lotion to several body regions (neck/shoulders, arms, back, legs/knee, n=5 per body region) for 1 week where dosing occurred in the morning and evening to address muscle tension/camps and/or pain.

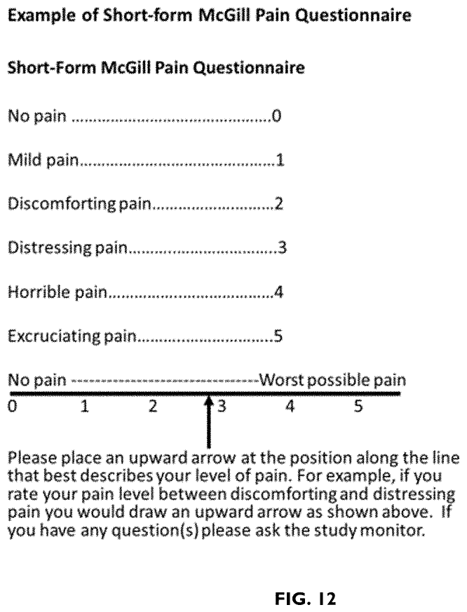

As shown in FIG. 12 there were 5 general ratings for the self-perception of pain from no pain=0 to excruciating pain=5 using the McGill pain questionnaire.

Results:

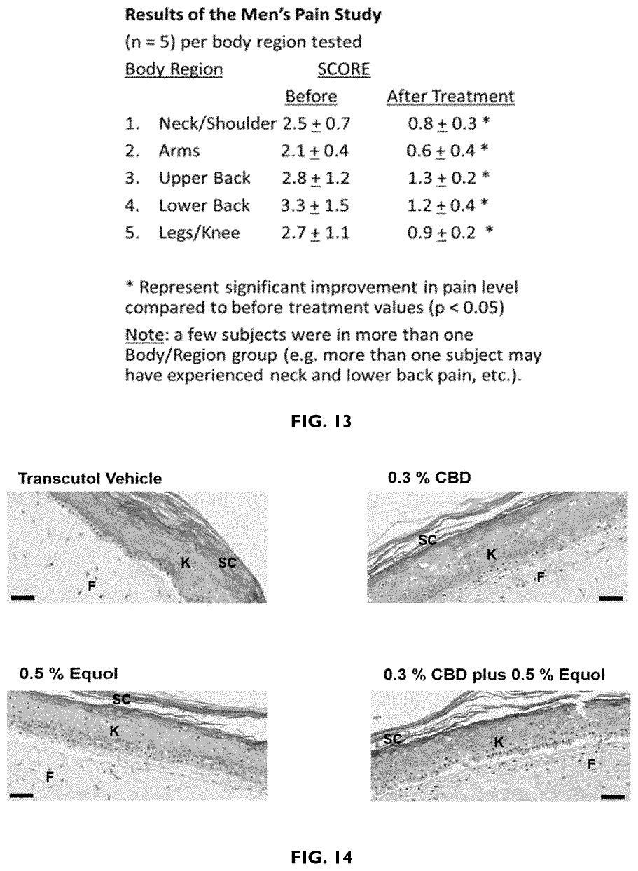

FIG. 13 displays the results of men's pain study.

As reported by the subjects, within 20 to 30 minutes following the application of the CBD/equol treatment there was a significant decrease in muscle pain/tension or cramping (via self-reporting surveys) as determined by statistical analysis. When the CBD/equol treatment was stopped for 1-3 days the muscle pain/tension/cramps returned, which were effectively treated when the topical CBD/equol treatment was resumed. There was a significantly greater effect when CBD and equol were combined, which yielded surprising results compared to when CBD or equol was used alone (as determined by statistical analysis).

Furthermore, subsequent clinical studies demonstrated therapeutic benefits in the management of pain where the treatment combinations that yielded significantly greater results to that in FIG. 13 included CBD and/or polyphenolic (equol) also with vitamin C and coenzyme Q10 mixtures, as determined by statistical analysis.

### Example 2: Human Skin Gene Expression Analysis

In human skin gene expression analysis full epidermal/dermal thickness human skin cultures were used.

The skin cultures were treated with: (a) transcutol vehicle; (b) 0.3% CBD; and (c) 0.5% equol and 0.3% CDB plus 0.5% equol.

In FIG. 14, the photomicrographs (@40× magnification) of the full epidermal/dermal thickness human skin cultures are displayed. All treatment slides (transcutol vehicle, 0.3% CBD, 0.5% equol and 0.3% CDB plus 0.5% equol) displayed intact and healthy epidermal (stratum corneum and keratinocytes) and dermal (fibroblast) components, and epidermal/dermal borders (FIG. 14).

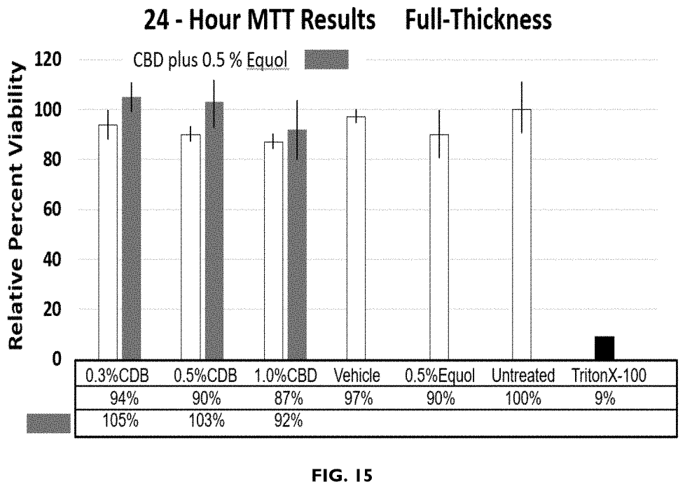

Validation of the full thickness epidermal/dermal human skin cultures cell viability by MTT assay is shown in FIG. 15.

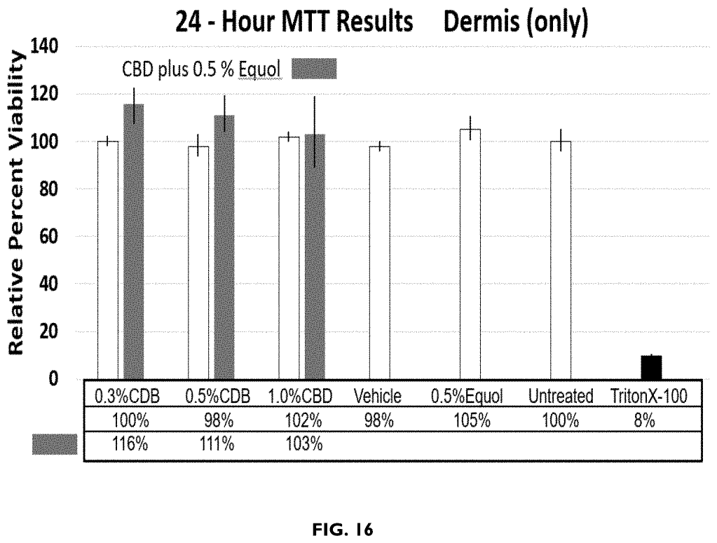

CBD alone, or equol alone, and 0.3% CBD and 0.5% equol showed the relative percent cell viability around 100%. Also, validation of the dermal layer (only) of the human skin cultures viability by MTT assay demonstrated that CBD alone, or equol alone, and 0.3% CBD and 0.5% equol had relative percent cell viabilities around 100% (FIG. 16).

Having established the human skin cultures proper and intact histology and cellular viability the treatment with a combination of CBD (at 0.3%) and racemic equol (at 0.5%) to epidermal full-thickness dermal cultures were performed using gene microarray analysis as reported elsewhere (Lephart, 2019). After 24 hours exposure to the 0.3% CDB and 0.5% equol treatment and the quantification of the gene expression levels for 136 skin biomarkers this resulted in surprising results where the combination CBD and racemic equol treatment demonstrated a synergistic, pleiotropic and entourage effects on various biological factors to significantly improve the dermal parameters (such as acne regulation, anti-aging, antioxidant, cell renewal, circadian rhythm regulation, epidermal barrier function, extracellular matrix breakdown protection, extracellular matrix integrity protection, pain and anti-inflammatory properties, pigmentation regulation tissue integrity and cell/skin remodeling and wound healing) that were not observed when the CBD or racemic equol treatments were administered alone (Table 4). For example, 94 skin genes out of 136 genes=70% of the skin genes tested yielded significant results (Table 4). [4 replicates per gene biomarker; statistical analysis via RQ method].

TABLE 4
Human Skin Gene Analysis: Gene Expression (fold-change) Influenced by the Combination
of 0.3% CBD plus 0.5% Equol, which resulted in 2-fold or greater change compared
to Vehicle Control Values (no symbol) and at least a 2-fold significantly different
(stimulation or inhibition) compared to 0.3% CBD or 0.5% Equol alone (# symbol).
Skin cell type: F = Fibroblast, K = Keratinocyte.

| | | | |
|---|---|---|---|
| | | | Skin |
| | Fold- | | cell |
| Gene Name | change | Skin Function | type |

ACNE REGULATION

| | | | |
|---|---|---|---|
| PPARG: Peroxisome Proliferator- | 3.36 | Pro-pigmentation; Anti- | F & K |
| Activated | | Proliferative; Anti-Psoriatic | |
| PSAP1: Prosaposin Like 1 | −3.77 # | Anti-Acne; Anti-Inflammatory | K |
| TRIB3: Tribbles Pseudo-kinase 3 | 3.92 | Anti-Acne; Anti-Inflammatory | F & K |

ANTI-AGING

| | | | |
|---|---|---|---|
| ADH1B: Alcohol Dehydrogenase 1B | 8.93 | Alcohol metabolism; Skin | F & K |
| (Class 1) | | blood Flow | |
| ELN: Elastin | 10.55 # | Anti-Aging (elasticity) | F & K |
| COL1A1: Collagen type 1 | 2.07 | Structural protein in | F |
| alpha 1 | | dermal matrix | |
| PPARGC1A: PPARG Coactivator 1 | 3.01 | ↑ Mitochondrial Biosynthesis; | F & K |
| alpha | | Pro-pigmentation | |
| SDR16C5: Short Chain | 4.42 # | Anti-psoriatic; Vitamin | K |
| Dehydrogenase/Reductase Family | | A & retinoic signaling | |
| 16C member 5 | | | |
| GSDMC: Gasdermin C | −2.84 | Extracellular Matrix Integrity | K |
| | | (↓ MMP expression); | |
| | | Anti- Photoaging | |

ANTI-OXIDANT

| | | | |
|---|---|---|---|
| AHR: Aryl Hydrocarbon Receptor | 2.67 | Phytochemicals bind & | F & K |
| | | Activate Nrf2 & ↑ | |
| | | Other antioxidants | |
| CAT: Catalase | 2.54 # | Anti-oxidant | F & K |
| DUOX1: Dual Oxidase 1 | −4.10 | Anti-apoptotic; anti- | F & K |
| | | inflammatory & Antioxidant | |
| DUOX1 | −2.63 # | Anti-inflammatory | F & K |
| EGLN3: Egl-9 Family Hypoxia | 4.29 # | ↑ Degradation of | F & K |
| Inducible Factor 3 | | hypoxia | |
| HAL: Histidine Ammonia Lyase | 6.10 # | Anti-oxidant; wound healing; | F & K |
| | | Anti-inflammatory | |
| HMOX1: Heme Oxygenase 1 | 3.48 # | Anti-oxidant; wound healing; | F & K |
| | | Anti-inflammatory | |
| MT1A: Metallothionein 1A | 3.58 # | Anti-oxidant | F & K |
| MT1G: Metallothionein 1G | 35.40 # | Anti-oxidant | F & K |
| NFE2L3: Nuclear Factor | −2.53 | Pro-survival; Anti- | F & K |
| Erythroid Like 3 | | Proliferative; Anti- | |
| | | Oxidant | |
| SLC30A1: Solute Carrier Family | 3.55 | Anti-inflammatory; | F & K |
| 30 Member 1 | | Antioxidant | |
| TXNRD1: Thioredoxin Reductase 1 | 7.71 | Protects Against | F & K |
| | | Free Radicals; | |
| | | Oxidative Stress & | |
| | | UV damage | |
| TXNRD1 | 4.09 # | Protects Against | F & K |
| | | Free Radicals; | |
| | | Oxidative Stress & | |
| | | UV damage | |

CELL RENEWAL

| | | | |
|---|---|---|---|
| CALM5: Calmodulin 5 | 5.22 # | Cell Growth & Renewal | K |
| CASP3: Caspase 3 | 2.84 | Cell Growth & Renewal | F & K |
| CASP14: Caspase 14 | 4.11 # | Cell Growth & Renewal | K |
| IGFL3: Insulin Growth Factor- | 5.33 # | Cell Growth & Renewal | K |
| Like Family Member 3 | | | |
| KRT5: Keratin 5 | −2.34 | Anti-pigmentation; Anti- | F & K |
| | | inflammatory | |
| KRT23: Keratin 23 | 11.13 # | Cell Renewal & Regeneration | K |
| S100A7: Anti-microbial | 7.22 # | Barrier Function; | K |
| Protein 7 | | Anti-psoriatic | |

CIRCADIAN RHYTHM REGULATION

| | | | |
|---|---|---|---|
| CIRT: Circadian Associated | −2.21 # | Anti-Proliferative | F & K |
| Repressor of Transcription | | | |
| CLOCK: Clock Circadian | 2.27 | DNA repair; Activates | K |
| Regulator | | PER & CRY; | |
| | | ↑ Hydration by AQP3; | |
| | | ↑Nutrient & | |
| | | Ingredients for Cell | |
| | | Repair for | |
| | | ↑ Barrier Function | |
| CRY1: Cryptochrome | 2.79 | CLOCK Regulator; | K |
| Circadian Regulator 1 | | Skin Firmness & | |
| | | Elasticity; ↓ Oxidative | |
| | | Stress | |
| NOCT: Nocturnin | −2.68 | Epidermal Homeostasis; Inverse | K |
| | | Expression to CLOCK | |
| NOCT | −3.13 # | Epidermal Homeostasis; Inverse | K |
| | | Expression to CLOCK | |
| PER2: Period Circadian Regulator 2 | 2.79 | CLOCK Regulator; | K |
| | | Modulate Hair Growth; | |
| | | ↓ Oxidative Stress | |
| PER2 | 2.38 # | CLOCK Regulator: | K |
| | | Modulate Hair Growth; | |
| | | ↓Oxidative Stress | |
| RORA: RAR Related Orphan | −3.05 # | ↓ Acne | F & K |
| Receptor A | | | |

EPIDERMAL BARRIER

| | | | |
|---|---|---|---|
| CST6: Cystatin E/M | 6.06 # | Barrier Integrity; Hydration | K |
| DNASE1L2: Deoxyribonuclease | 3.31 # | Barrier Integrity | K |
| 1 Like 2 | | | |
| TGM5: Transglutaminase 5 | 12.63 # | Barrier Function & Hydration | K |

EXTRACELLULAR MATRIX BREAKDOWN

| | | | |
|---|---|---|---|
| KLK5: Kallikrein | −4.67 # | Proteolytic/Digestion: | K |
| Related Peptidase 5 | | Collagen; Fibronectin & | |
| | | Laminin | |
| KLK7: Kallikrein | −5.31 # | IL4 & IL14; ↑ KLK | K |
| Related Peptidase 7 | | which ↓ Flaggrin | |
| MMP10: Matrix Metalloproteinase | −3.57 # | Anti-Aging; Retention of | F & K |
| 10 | | Collagen & Elastin | |
| SERPINB3: Serpin Family B | −16.20 | Anti-Psoriatic: | F & K |
| Member 3 | | Anti-Inflammatory | |
| SERPINB3 | 4.31 # | Barrier Integrity; | F & K |
| | | UV Protection | |
| SPINK5: Serine Peptidase Inhibitor | 6.25 # | Encodes LEKT1 | K |
| Kazal Type 5 | | which inhibits KLK5 & KLK7 | |
| ST14: Suppression | −6.94 | Anti-Inflammatory | K |
| Tumorigenicity 14 (Matriptase) | | | |
| ST14 | −5.03 # | Anti-Inflammatory | K |
| DMKN: Dermokine | −12.01 | Anti-Psoriatic: | K |
| | | Anti-Inflammatory | |

EXTRACELLULAR MATRIX INTEGRITY

| | | | |
|---|---|---|---|
| DSC1: Desmocollin 1 | 6.29 # | Barrier Function | K |
| DSG1: Desmoglein 1 | 3.03 # | Barrier Function | K |
| SERPINH1: Serpin | 7.06 | Wound Healing | F & K |
| Family H Member 1 | | | |
| TIMP1: Tissue Inhibitor of Matrix | 2.83 | Anti-Aging (↓ MMPs) | F & K |
| Metalloproteinase 1 | | | |

PAIN & INFLAMMATION

| | | | |
|---|---|---|---|
| ANXA9: Annexin A9 | −19.16 # | Anti-Inflammatory | K |
| ARG1: Arginase 1 | −4.25 # | Anti-Inflammatory | K |
| CSF2: Colony | −7.78 # | Anti-Inflammatory | F & K |
| Stimulating Factor 2 | | | |
| IL10: Interleukin 10 | 2.14 | Anti-Inflammatory | F & K |
| IRF1: Interferon | 6.57 | Tumor-Suppressor | F & K |
| Regulatory Factor 1 | | | |
| IRF1 | 2.23 # | Anti-Inflammatory | F & K |
| IFNA1: Interferon Alpha 1 | 2.10 # | Anti-Inflammatory | K |
| IL24: Interleukin 24 | 3.63 | Anti-Proliferative | F & K |
| TLR2: Toll Like Receptor 2 | −10.25 | Anti-Inflammatory; Anti-Acne | F & K |
| TNGSF10: Tumor | −8.52 | Anti- Psoriatic | F & K |
| Necrosis Factor | | | |
| Superfamily Member 10 | | | |
| IL18: Interleukin 18 | −8.39 | Anti-Inflammatory | K |
| ILI8 | −2.40 # | Anti-Inflammatory | K |
| IL20RA: Interleukin 20 Receptor | −6.75 | Anti-Inflammatory; | K |
| Subunit Alpha | | ↓ Pigmentation | |
| IL20RA | −2.62 # | Anti-Inflammatory | K |
| IL20RB: Interleukin 20 Receptor | −3.28 | Anti-Psoriatic; | K |
| Subunit Beta | | Anti-Inflammatory | |
| SERPINA12: Serpin | 17.23 # | Anti-Inflammatory; | K |
| Family A Member 12 | | Anti-microbial | |
| WFDC5: WAP Four- | −6.05 # | Anti-Inflammatory; | K |
| Disulfide Core Domain 5 | | Anti-psoriatic | |

PIGMENTATION

| | | | |
|---|---|---|---|
| ADRB2: Adrenoceptor Beta 2 | −3.47 | Anti-Pigmentation | F & K |
| ALOX12B: Arachidonate 12- | −33.74 | Anti-Pigmentation; | K |
| Lipoxygenase 12R Type | | Whitening & Skin Brightening | |
| BMP6: Bone Morphogenetic | 9.63 | Anti-Pigmentation; | F & K |
| Protein 6 | | Anti-Scarring | |
| CRYBG1 (AIM1): | −2.75 # | Anti-Pigmentation | F & K |
| Crystallin Beta-Gamma Domain | | | |
| Containing 1 | | | |
| EDNRB: Endothelin | 3.43 | Pro-pigmentation | F & K |
| Receptor Type B | | | |
| KIT: KIT Proto-Oncogene | −2.52 | Anti-Pigmentation | F & k |
| Receptor Tyrosine Kinase | | | |
| MC1R: Melanocortin 1 Receptor | −4.87 | Anti-Pigmentation | F & K |
| MITF: Melanogenesis Associated | −2.66 # | Anti-Pigmentation | F & K |
| Transcription Factor | | | |
| STAP2: Signal Transducing | −4.27 | Anti-Pigmentation | F & K |
| Adaptor Family Member 2 | | | |

SKIN HYDRATION

| | | | |
|---|---|---|---|
| ALOX12B: Arachidonate | 10.26 # | Barrier Integrity & Hydration | K |
| 12-Lipoxygenase 12R Type | | | |
| ALOXE3: Arachidonate Lipoxygenase | 6.58 # | Barrier Integrity & Hydration | K |
| AQP9: Aquaporin 9 | 7.74 # | Hydration | K |
| EPHX3: Epoxide Hydrolase 3 | 3.89 # | Barrier Integrity & Hydration | K |

TISSUE INTEGRIETY/REMODELING

| | | | |
|---|---|---|---|
| ABCG4: ATP Binding Cassette Subfar | 11.16 # | Cell Survival & | F & K |
| G Member 4 | | Wound Healing | |
| CD36: CD 36 Molecule | 4.50 # | Collagen Binding; | K |
| | | Cell Adhesion | |
| CDSN: Corneodesmosin | 7.32 # | Barrier Integrity | K |
| FLG2: Filaggrin Family Member 2 | 9.46 # | Keratin Integrity; | K |
| | | Differentiation | |
| KRT1: Keratin 1 | 3.31 # | Barrier Function | K |
| KRT10: Keratin 10 | 2.64 # | Barrier Function | K |

WOUND HEALING

| | | | |
|---|---|---|---|
| EFNA3: Ephrin A3 | −6.77 | ↓ Scar Formation | K |
| F3: Coagulation Factor III Tissue | 2.24 # | ↑ Wound Healing | F & K |
| Factor | | | |
| FGF2: Fibroblast Growth Factor 2 | 8.08 | Growth Factor & | K |
| | | ↑ Wound Healing | |
| MT1F: Metallothinein 1 F | 5.09 # | Anti-oxidant & | F & K |
| | | ↑ Wound Healing | |
| PKP1: Plakophilin 1 | −4.93 | ↓ Scar Formation | K |

Referring to Table 4, 94 genes out of 136 genes=approximately 70% of the genes tested yielded surprising and significant results (greater than a 2-fold change) compared to control values and/or significantly greater than compared to CDB or equol alone (as determined by statistical analysis).

Notably, in a further series of experiments where the other natural active ingredients, such as collagen, vitamin C, vitamin E, Zinc, grape seed extract, niacinamide, hydroxy acids, hyaluronic acid, or coenzyme Q10 were tested in combination with CBD and/or polyphenolic compounds, the obtained results that displayed significant unexpected findings that were better in than CBD or polyphenolic treatments alone, (as determined by statistical analysis) see the below-human clinical skin studies.

### Example 3: Human Clinical Skin Study I

To examine the influence of CBD and equol on human skin a clinical study was performed in 40 adult female subjects. The treatment was applied topically twice a day. The formulations used in the study were skin topical formulation 1 (STF1) or skin topical formulation 2 (STF2), shown in Table 2.

Table 5 shows the demographic characteristics as far as the age, ethnicity and Fitzpatrick skin type of the female subjects are displayed. The average age was 55.8 years+6.2 years (Table 5), representing women, some of which were postmenopausal (63%).

TABLE 5
Summary Demographic Characteristics, Human Skin Study I.
Table 5. Summary of Demographic Characteristics - Study I
Forty (40) female subjects completed the study

| | | |
|---|---|---|
| Age (Years) | Average ± | 55.8 ± 6.2 |
| | Standard Deviation | |
| | Minimum | 34.9 |
| | Maximum | 66.4 |
| Ethnicity | Caucasian | 26 (65.0%) |
| | Chinese | 2 (5.0%) |
| | Japanese | 12 (30.0%) |
| Fitzpatrick | Type I | 10 (25.0%) |
| Skin Type | Type II | 20 (50.0%) |
| | Type III | 10 (25.0%) |

Fitzpatrick Skin Classification (Skin Photo-type) is
based on a person's complexion and responses to sun exposure:

| | |
|---|---|
| Type I | Always burns easily; never tans |
| Type II | Always burns easily; tans minimally |
| Type III | Burns moderately; tans gradually |
| Type IV | Burns minimally; always tans well |
| Type V | Rarely burns; tans profusely |
| Type VI | Never burns; deeply pigmented |

The Clinical Grading included fine lines, coarse wrinkles, texture and smoothness, tone and resiliency, pore size, radiation and overall appearance of the skin (Table 6) that was performed by dermatologist(s) at week 2, week 4, week 8 and finally at week 12 at the end of the study (this clinical grading was used in the first and second clinical skin studies.)

TABLE 6
Human Skin Study: Clinical Grading - Efficacy of
Evaluations (utilized in Studies I and II).
Table 6. Clinical Grading (Utilized In Studies I and II)
Efficacy Evaluations
The following efficacy parameters were assessed using a
Griffith's 10-point scale (0 to 9) where 0 = none,
1 to 3 = mild, 4 to 6 = moderate and 7 to 9 = severe.
The scale parameters are listed below:

| Parameter | 0= | 9= |
|---|---|---|
| Fine Lines- | None | Numerous, deep fine |
| Periocular & | | lines |
| Overall facial | | |
| Coarse Wrinkles- | None | Numerous, deep fine |
| Periocular & | | lines |
| Overall facial | | |
| Texture & | Smooth | Rough/coarse to the |
| Smoothness | | touch |
| Firmness | Firm, tight appearing skin | Loose appearing skin |
| Tone & | Even, healthy skin color | Uneven, discolored |
| Resiliency | | appearance |
| Pore Size | Small, even skin appearance | Large, uneven skin |
| | | appearance |
| Radiance | Radiant, luminous or glowing | Dull/matte and/or |
| | | sallow |
| Overall | Excellent | Poor |
| Appearance | | |

The results are summarized in Table 7.

As shown in Table 7, fine lines improved 100%, coarse wrinkles 84%, in the periocular area by week 12. In the face area, fine lines improved 100%, coarse wrinkles 78%, texture and smoothness 100%, firmness 77%, tone and resiliency 98%, pore size 89%, radiance 99% and overall appearance 100% by week 12 (Table 7).

TABLE 7
Human Skin Study: Results of Clinical Grading, Clinical Study I.
Table 7. Results of Clinical Grading-Study I

| | | | |
|---|---|---|---|
| Week 2 | Week 4 | Week 8 | Week 12 |

Efficacy- Periocular Area - % improved over baseline

| | | | | |
|---|---|---|---|---|
| Fine Lines- | 84% | 92% | 100% | 100% |
| Coarse Wrinkles- | 35% | 61% | 73% | 84% |

Efficacy - Face Area - % improved over baseline

| | | | | |
|---|---|---|---|---|
| Fine Lines- | 76% | 89% | 98% | 100% |
| Coarse Wrinkles- | 18% | 44% | 63% | 78% |
| Texture & Smoothness- | 88% | 100% | 100% | 100% |
| Firmness- | 29% | 44% | 62% | 77% |
| Tone & Resiliency- | 46% | 77% | 93% | 98% |
| Pore Size- | 43% | 64% | 79% | 89% |
| Radiance- | 79% | 92% | 95% | 99% |
| Overall Appearance- | 61% | 83% | 98% | 100% |

The parameters were quantified for each subject taken at Baseline, Week 2, Week 4, Week 8, and Week 12. The data were analyzed by ANOVA followed by pairwise comparisons. For each parameter there was a significant increase (% improvement over baseline) for each time interval, p < 0.05.

The results of the VISIA digital image analysis (which is a multi-point positioning system that captures live images for different facial parameters) of the face area at 12 weeks showed that pores improved 66%, porphyrins 29%, spots 46%, texture 67%, UV spots 52% and wrinkles by 82%. Results of the VISIA analysis are summarized in Table 8.

TABLE 8
Human Skin Study: Results of VISIA Analysis, Clinical Study I.
Table 8. Results of VISIA Image Analysis-Study I
Efficacy- Facial Area- % improved over baseline

| | Week 2 | Week 4 | Week 8 | Week 12 |
|---|---|---|---|---|
| Pores- | 54% | 57% | 60% | 66% |
| Porphyrins- | 14% | 19% | 25% | 29% |
| Spots- | 28% | 37% | 42% | 46% |
| Texture- | 51% | 57% | 63% | 67% |
| UV Spots- | 18% | 26% | 38% | 52% |
| Wrinkles- | 42% | 58% | 69% | 82% |

VISIA digital images of each subject taken at Baseline, Week 2, Week 4, Week 8, and Week 12 were analyzed using the VISIA complexion software. The data were analyzed by ANOVA followed by pairwise comparisons. For each parameter at each time interval there was a significant increase (% improvement over baseline), p < 0.05.

The results of the self-assessment questionnaire for the facial area at 12 weeks showed the appearance of fine lines and wrinkles improved 91%, medium and deep lines and wrinkles 88%, eyes (crow's feet) 87% and smile and frown lines 84%. The results of the self-assessment questionnaire are shown in Table 9.

TABLE 9
Human Skin Study: Results of Self-Assessment
Questionnaire, Clinical Study I.
Table 9. Results of Self-Assessment Questionnaire Analysis-Study I
Efficacy- Facial Area- % improved over baseline

| WRINKLES | Week 2 | Week 4 | Week 8 | Week 12 |
|---|---|---|---|---|
| Appearance of | 56% | 74% | 85% | 91% |
| Fine Lines/Wrinkles | | | | |
| Appearance of | 58% | 77% | 82% | 88% |
| Medium/Deep Lines/ | | | | |
| Wrinkles- | | | | |
| Eyes (Crow's Feet) | 55% | 74% | 83% | 87% |
| Smile/Frown Lines- | 49% | 70% | 81% | 84% |

Each subject quantified each parameter at Baseline, Week 2, Week 4, Week 8, and Week 12. The data were analyzed by ANOVA followed by pairwise comparisons. For each parameter at each time interval there was a significant increase (% improvement over baseline), p < 0.05.

Table 10, displays the results of the self-assessment questionnaire for the facial area of skin attributes such as, overall skin firmness improved 88%, firmness around the eyes 84%, sin sensitivity 72%, even skin tone 86%, skin brightness 87%, noticeability of pores 77%, skin hydration 93%, skin spots (discoloration) by 79% and skin softness 88% by week 12. The results are summarized in Table 10.

TABLE 10
Results of Self-Assessment Questionnaire Analysis-Study I.
Table 10. Results of Self Assessment Questionnaire Analysis-Study I
Efficacy- Facial Area - % improved over baseline

| SKIN ATTRIBUTES | Week 2 | Week 4 | Week 8 | Week 12 |
|---|---|---|---|---|
| Overall Skin Firmness- | 44% | 64% | 83% | 88% |
| Firmness around eyes- | 39% | 58% | 78% | 84% |
| Skin Sensitivity- | 46% | 54% | 63% | 72% |
| Skin Tone (Evenness)- | 62% | 73% | 79% | 86% |
| Skin Brightness- | 59% | 74% | 82% | 87% |
| Noticeability of pores- | 37% | 57% | 63% | 77% |
| Skin Hydration- | 56% | 69% | 81% | 93% |
| Skin Spots (discoloration)- | 62% | 73% | 77% | 79% |
| Skin Softness- | 64% | 77% | 83% | 88% |

Each subject quantified each parameter at Baseline, Week 2, Week 4, Week 8, and Week 12. The data were analyzed by ANOVA followed by pairwise comparisons. For each parameter at each time interval there was a significant increase (% improvement over baseline), p < 0.05.

Table 11 shows the responses of the subjects tested for product attributes, efficacy and tolerance: overall positive opinion of the product 94%, ease of application 97%, compatibility with makeup 92%, mildness/gentleness 95%, overall sensory experience 91%, speed of effects or results of using the product 87%, and range of effects 88% by week 12. The results are summarized in Table 11.

TABLE 11
Results of CHI-Squared Analysis for Self-Assessment Questionnaires -
for Product Attributes, Efficacy and Tolerance - Study I.
Table 11. Results of CHI-Squared Analysis for Self-Assessment
Questionnaires - for Product Attributes, Efficacy and Tolerance-Study I
Product Attributes- % of subjects responded positively

| | Week 2 | Week 4 | Week 8 | Week 12 |
|---|---|---|---|---|
| Overall Opinion- | 71% | 82% | 89% | 94% |
| Ease of Application- | 89% | 91% | 94% | 97% |
| Compatibility with make-up | 76% | 88% | 93% | 92% |
| Mildness/Gentleness- | 87% | 91% | 93% | 95% |
| Overall Sensory Experience | 77% | 85% | 88% | 91% |
| Speed of Effects/Results- | 76% | 79% | 85% | 87% |
| Range of Effects- | 72% | 79% | 84% | 88% |

All data parameters were statistically significant (p < 0.05) proportion of subjects responded positively.

Results of CHI-squared analysis for self-assessment questionnaires—for product attributes, efficacy and tolerance are summarized in Table 12. Specifically, the product containing CDB and equol had attributes that made the subjects skin: look younger 84%, feel younger 87%, look healthier 89%, feels healthier 85% and during the study received positive comments from associates about their skin 83% by week 12 (Table 12).

TABLE 12
Results of CHI-Squared Analysis for Self-Assessment Questionnaires -
For Product Attributes, Efficacy and Tolerance.
Table 12. Results of CHI-Squared Analysis for Self-Assessment
Questionnaires - for Product Attributes, Efficacy and Tolerance-Study I
Product Attributes- % of subjects responded positively

| | Week 2 | Week 4 | Week 8 | Week 12 |
|---|---|---|---|---|
| My Skin Looks Younger- | 71% | 76% | 81% | 84% |
| My Skin Feels Younger- | 69% | 71% | 84% | 87% |
| My Skin Looks Healthier- | 74% | 82% | 86% | 89% |
| My Skin Feels Healthier- | 70% | 77% | 83% | 85% |
| During the Study, I received | 63% | 77% | 78% | 83% |
| Positive comments from | | | | |
| People about my skin- | | | | |

All data parameters were statistically significant (p < 0.05) proportion of subjects responded positively.

Table 13 displays the tabulations of years younger responses for self-assessment questionnaires by week 12 about—how many years younger does my skin feel? 4.46 years to how many years younger does my skin radiance look? 5.12 years (among 5 different measured parameters that were analyzed).

TABLE 13
Tabulations of Years Younger Responses For Self-
Assessment Questionnaires by Week 12-Study I

| | |
|---|---|
| How many years younger does my skin feel? | 4.46 (years) |
| How many years younger does my skin look? | 3.96 (years) |
| How many years younger does my skin firmness look? | 3.91 (years) |
| How many years younger does my skin tone look? | 4.56 (years) |
| How many years younger does my skin radiance look? | 5.12 (years) |

In summary for the human skin clinical study 1, the female subjects that used the combination CDB and equol topical treatments for up to 12 weeks showed a significant improvement in all the skin health parameters of the study. The results are summarized in Tables 7 through 13 above.

Subsequent clinical studies demonstrated that when the other natural active ingredients, such as collagen, vitamin C, vitamin E, Zinc, grape seed extract, niacinamide, hydroxy acids, hyaluronic acid, or coenzyme Q10 were tested in combination with CBD and/or polyphenolic compounds the obtained results yielded significant surprising findings that were better in than the CBD or polyphenolic treatments alone (via statistical analysis). Examples of such embodiments can be substantially derived from formulations displayed in Table 2 for skin/topical applications.

For example, the natural combinations that yielded some of the best unexpected results included equol along with vitamin C, grape seed extract and hyaluronic acid mixtures or equol plus vitamin C and hyaluronic acid were tested in the second clinical skin study. As stated above, examples of such embodiments can be substantially derived from formulations displayed in Table 2 for skin/topical applications, which were determined by statistical analysis.

### Example 4. Human Clinical Skin Study II

In the second clinical skin study, 42 women completed the study, which had Glogua Aging II to III (mild to moderate wrinkling) where 30 out of the 42 (i.e., 71% of the women) were amenorrheic for at least approximately 4 years (i.e., postmenopausal). The combination treatment mixture (see below in the next paragraph) was applied twice per day (morning and evening). The summary demographic characteristics are displayed in Table 14.

TABLE 14
Summary of Demographic Characteristics - Study II
Forty (42) female subjects completed the study

| | | |
|---|---|---|
| Age (Years) | Average ± Standard Deviation | 57.3 ± 7.3 |
| | Minimum | 40.1 |
| | Maximum | 70.2 |
| Ethnicity | Caucasian | 23 (55.0%) |
| | Chinese | 4 (10.0%) |
| | Japanese | 15 (35.0%) |
| Fitzpatrick | Type I | 12 (29.0%) |
| Skin Type | Type II | 18 (42.0%) |
| | Type III | 12 (29.0%) |

Fitzpatrick Skin Classification (Skin Photo-type) is based on a
person's complexion and responses to sun exposure:

| | |
|---|---|
| Type I | Always burns easily; never tans |
| Type II | Always burns easily; tans minimally |
| Type III | Burns moderately; tans gradually |
| Type IV | Burns minimally; always tans well |
| Type V | Rarely burns; tans profusely |
| Type VI | Never burns; deeply pigmented |

One-half of the women in the second clinical skin study were tested with a skin topical formulation 6 (STF6), while the other half of the women were tested with a skin topical formulation 7 (STF7), as shown in Table 2 above.

The STF6 treatment results were 8 to 13 percent better compared to the STF7 for many of the skin parameters tested, however, since the overall quantified skin results were similar between the two treatment groups the data was combined and presented herein as the clinical skin study II data.

The results of the clinical grading from the second clinical skin study are shown in Table 15.

TABLE 15
Results of Clinical Grading- Study II

| | | | |
|---|---|---|---|
| Week 2 | Week 4 | Week 8 | Week 12 |

Efficacy- Periocular Area - % improved over baseline

| | | | | |
|---|---|---|---|---|
| Fine Lines- | 87% | 95% | 100% | 100% |
| Coarse Wrinkles- | 44% | 69% | 82% | 88% |

Efficacy - Face Area - % improved over baseline

| | | | | |
|---|---|---|---|---|
| Fine Lines- | 79% | 93% | 100% | 100% |
| Coarse Wrinkles- | 24% | 51% | 76% | 85% |
| Texture & Smoothness- | 93% | 100% | 100% | 100% |
| Firmness- | 36% | 54% | 71% | 83% |
| Tone & Resiliency- | 56% | 81% | 95% | 100% |
| Pore Size- | 49% | 73% | 84% | 93% |
| Radiance- | 83% | 94% | 100% | 100% |
| Overall Appearance- | 69% | 87% | 100% | 100% |

The parameters were quantified for each subject taken at Baseline, Week 2, Week 4, Week 8, and Week 12. The data were analyzed by ANOVA followed by pairwise comparisons. For each parameter there was a significant increase (% improvement over baseline) for each time interval, p < 0.05.

As shown in Table 15, the results were significantly greater for: a) coarse wrinkles in the periocular area, and in the facial area, b) coarse wrinkles, c) firmness, and d) pore size, as compared to the first clinical skin study that used the combination of CDB and equol as the treatment (see STF1 and/or STF2 in Table 2 for formulation examples).

The results of the VISIA imaging analysis from the second clinical study are shown in Table 16.

TABLE 16
Results of VISIA Image Analysis-Study II
Efficacy- Facial Area- % improved over baseline

| | Week 2 | Week 4 | Week 8 | Week 12 |
|---|---|---|---|---|
| Pores- | 60% | 66% | 71% | 77% |
| Porphyrins- | 19% | 23% | 30% | 35% |
| Spots- | 34% | 46% | 53% | 60% |
| Texture- | 60% | 65% | 70% | 72% |
| UV Spots- | 23% | 29% | 46% | 61% |
| Wrinkles- | 49% | 67% | 77% | 89% |

VISIA digital images of each subject taken at Baseline, Week 2, Week 4, Week 8, and Week 12 were analyzed using the VISIA complexion software. The data were analyzed by ANOVA followed by pairwise comparisons. For each parameter at each time interval there was a significant increase (% improvement over baseline), p < 0.05.

Referring to Table 16, the efficacy of the percent improvement over baseline in the facial area for: a) pores at 77%, b) porphyrins at 35%, c) spots at 60%, d) texture at 72%, e) UV spots at 61% and f) wrinkles at 89% using the combination of the skin topical formulation STF 6 and STF7 as substantially described in Table 2 were significantly greater as compared to the findings from the first clinical skin study.

The results of the self-assessment questionnaire analysis from the second clinical study are shown in Table 17.

TABLE 17
Results of Self-Assessment Questionnaire Analysis-Study II
Efficacy- Facial Area- % improved over baseline

| WRINKLES | Week 2 | Week 4 | Week 8 | Week 12 |
|---|---|---|---|---|
| Appearance of | 63% | 79% | 88% | 94% |
| Fine Lines/Wrinkles | | | | |
| Appearance of | 66% | 85% | 89% | 92% |
| Medium/Deep Lines/ | | | | |
| Wrinkles- | | | | |
| Eyes (Crow's Feet) | 59% | 78% | 87% | 91% |
| Smile/Frown Lines- | 54% | 78% | 85% | 89% |

Each subject quantified each parameter at Baseline, Week 2, Week 4, Week 8, and Week 12. The data were analyzed by ANOVA followed by pairwise comparisons. For each parameter at each time interval there was a significant increase (% improvement over baseline), p < 0.05.

Referring to Table 17, the efficacy of the percent improvement over baseline in the facial area for: a) fine lines/wrinkles at 94%, b) medium to deep lines and wrinkles at 92%, c) crow's feet around the eyes at 91% and smile/frown lines at 89% using the combination of the skin topical formulation STF 6 and STF7 as substantially described in Table 2 were significantly greater as compared to the findings from the first clinical skin study (via statistical analysis).

The results of the self-assessment questionnaire analysis from the second clinical study are shown in Table 18.

TABLE 18
Results of Self Assessment Questionnaire Analysis-Study II
Efficacy- Facial Area - % improved over baseline

| SKIN ATTRIBUTES | Week 2 | Week 4 | Week 8 | Week 12 |
|---|---|---|---|---|
| Overall Skin Firmness- | 51% | 88% | 88% | 91% |
| Firmness around eyes- | 41% | 63% | 79% | 87% |
| Skin Sensitivity- | 53% | 81% | 72% | 80% |
| Skin Tone (Evenness)- | 71% | 78% | 83% | 89% |
| Skin Brightness- | 83% | 71% | 79% | 90% |
| Noticeability of pores- | 44% | 83% | 89% | 81% |
| Skin Hydration- | 62% | 70% | 85% | 95% |
| Skin Spots (discoloration)- | 69% | 75% | 83% | 84% |
| Skin Softness- | 89% | 82% | 87% | 92% |

Each subject quantified each parameter at Baseline, Week 2, Week 4, Week 8, and Week 12. The data were analyzed by ANOVA followed by pairwise comparisons. For each parameter at each time interval there was a significant increase (% improvement over baseline), p < 0.05.

Referring to Table 8, the self-assessment questionnaire analysis from the second clinical study that covered the efficacy of the percent improvement over baseline in the facial area for: a) overall skin firmness at 91%, b) firmness around the eyes at 87%, c) skin sensitivity at 80%, d) even skin tone at 89%, e) skin brightness at 90%, f) noticeability of pores at 81%, g) skin hydration at 95%, h) skin spots (discoloration) at 84% and i) skin softness at 92% using the combination of the skin topical formulation STF 6 and STF7 as substantially described in Table 2 and in paragraph [0112] above, which were significantly greater compared to the findings from the first clinical skin study (via statistical analysis).

The results of the CHI-squared analysis for self-assessment questionnaire for product attributes, efficacy and tolerance for the subjects that responded positively in the second clinical study (at the end of the treatment at 12 weeks) are shown in Table 19.

TABLE 19
Results of CHI-Squared Analysis for Self-Assessment Questionnaires -
for Product Attributes, Efficacy and Tolerance-Study II
Product Attributes- % of subjects responded positively

| | Week 2 | Week 4 | Week 8 | Week 12 |
|---|---|---|---|---|
| Overall Opinion- | 73% | 84% | 90% | 96% |
| Ease of Application- | 90% | 93% | 95% | 98% |
| Compatibility with make-up | 79% | 91% | 94% | 95% |
| Mildness/Gentleness- | 89% | 93% | 95% | 97% |
| Overall Sensory Experience | 79% | 87% | 91% | 93% |
| Speed of Effects/Results- | 80% | 83% | 87% | 89% |
| Range of Effects- | 76% | 84% | 87% | 90% |

All data parameters were statistically significant (p < 0.05) proportion of subjects responded positively.

The percent of subjects that responded positively to the product attributes included: a) overall opinion at 96%, b) ease of application at 98%, c) compatibility with make-up at 95%, d) mildness or gentleness at 97%, e) overall sensory experience at 93%, f) speed of effects or results perceived at 89%, and g) range of positive effects at 90% using the combination of the skin topical formulation STF 6 and STF7 as substantially described in Table 2 and in paragraph [0112] above, which were significantly greater compared to the findings from the first clinical skin study (except for ease of application and mildness or gentleness; via statistical analysis). Additional results of the CHI-squared analysis for self-assessment questionnaire of tested subjects for product attributes that responded positively in the second clinical study (at the end of the treatment at 12 weeks) are shown in Table 20.

TABLE 20
Results of CHI-Squared Analysis for Self-Assessment Questionnaires -
for Product Attributes, Efficacy and Tolerance-Study II
Product Attributes- % of subjects responded positively

| | Week 2 | Week 4 | Week 8 | Week 12 |
|---|---|---|---|---|
| My Skin Looks Younger- | 75% | 83% | 85% | 89% |
| My Skin Feels Younger- | 73% | 74% | 88% | 90% |
| My Skin Looks Healthier- | 79% | 84% | 89% | 91% |
| My Skin Feels Healthier- | 72% | 79% | 86% | 89% |
| During the Study, I received | 69% | 82% | 85% | 88% |
| Positive comments from | | | | |
| People about my skin- | | | | |

All data parameters were statistically significant (p < 0.05) proportion of subjects responded positively.

Referring to Table 20, four of the five measured parameters were significantly greater when using the combination of the skin topical formulation STF 6 or STF7, as substantially described in Table 2 as compared to the findings from the first clinical skin study that included: a) my skin looks younger at 89%, b) my skin feels younger at 90%, c) my skin feels healthier at 91% and during this study, I received positive comments from people about my skin at 88% (via statistical analysis).

Finally, the tabulation of years younger responses for the self-assessment questionnaires by week 12 in the second clinical study are displayed in Table 21.

TABLE 21
Tabulations of Years Younger Responses For Self-
Assessment Questionnaires by Week 12-Study II

| | |
|---|---|
| How many years younger does my skin feel? | 5.26 (years) |
| How many years younger does my skin look? | 4.86 (years) |
| How many years younger does my skin firmness look? | 4.81 (years) |
| How many years younger does my skin tone look? | 5.12 (years) |
| How many years younger does my skin radiance look? | 6.12 (years) |

Referring to Table 21, all five measured parameters: a) how many years younger does my skin feel at 5.26 years, b) how many years younger does my skin look? at 4.86 years, c) how many years younger does my skin firmness look? at 4.81, d) how many years younger does my skin tone look? at 5.12 and e) how many years younger does my skin radiance look? at 6.12 using the combination of the skin topical formulation STF 6 or STF7, as substantially described in Table 2 which were significantly greater as compared to the findings from the first clinical skin study (via statistical analysis).

When the data from the second clinical skin study was compared with data from the first clinical skin study, the results obtained in Tables 15 through 21 from the second study were significantly greater (via statistical analysis) than that shown in Tables 7 through 10 and Tables 12 and 13 from the first clinical skin study (Table 22). However, both the first and second clinical skin studies demonstrated surprising and unexpected results, due to the efficacy of the percent improvement of the quantified parameters over the 12-week topical dermal investigation in women. For example, when the results from prior clinical studies where an analog of resveratrol (i.e., 4′-acetoxy resveratrol or 4AR alone) or equol alone was tested (Naftolin & Lephart, 2021) were compared to the results from clinical skin study I or the results from clinical skin study II, there was a 30% significant increase on average among all of the skin parameters tested when compared to 4 AR results alone and there was a significant 44% increase in efficacy and when compared to the equol results alone (Table 22).

TABLE 22
Comparison of Results (Self-Assessment Questionnaire Analysis-Efficacy) from 4′-Acetoxy-Resveratrol (4AR, alone) at 1.0% vs.
Equol (alone) at 0.3% (prior Skin Study) vs. CDB + Polyphenol (Equol) (1^{st} Skin Study) vs. Polyphenol (Equol) plus Natural Ingredients
(2^{nd} Skin Study) (e.g., Grape Seed Extract, Vitamin C and Hyaluronic Acid or Vitamin C and Hyaluronic Acid) at 12 weeks

| SKIN | | | CBD + | Polyphenol (Equol) |
|---|---|---|---|---|
| ATTRIBUTES | 4AR | Equol | Polyphenol (Equol) (1^{st} Skin Study) | Plus Natural Ingredients (2^{nd} Skin Study) |
| Skin Firmness | 68% | 77% | 84% [23% ↑ over 4AR; 9% ↑ over Equol] | 91% [34% ↑ over 4AR; 18% ↑ over Equol] |
| Smoothness | 72% | 62% | 88% [22% ↑ over 4AR; 42% ↑ over Equol] | 100% [39% ↑ over 4AR; 61% ↑ over Equol] |
| Even Skin Tone | 83% | 70% | 86% [4% ↑ over 4AR; 23% ↑ over Equol] | 100% [21% ↑ over 4AR; 43% ↑ over Equol] |
| Frown Lines/ | 78% | 72% | 84% [8% ↑ over 4AR; 17% ↑ over Equol] | 89% [14% ↑ over 4AR; 24% ↑ over Equol] |
| Wrinkles | | | | |
| Radiance/ | 72% | 73% | 87% [21% ↑ over 4AR; 19% ↑ over Equol] | 100% [39% ↑ over 4AR; 37% ↑ over Equol] |
| Brightness | | | | |
| Pore Size | 64% | 51% | 77% [20% ↑ over 4AR; 51% ↑ over Equol] | 93% [45% ↑ over 4AR; 82% ↑ over Equol] |
| Spots/ | 74% | 55% | 79% [7% ↑ over 4AR; 44% ↑ over Equol] | 84% [14% ↑ over 4AR; 53% ↑ over Equol] |
| Discoloration | | | | |
| Hydration | 72% | 71% | 93% [29% ↑ over 4AR; 31% ↑ over Equol] | 95% [32% ↑ over 4AR; 34% ↑ over Equol] |
| | | | [17% average ↑ over 4AR; 30% average ↑ over Equol] | [30% average ↑ over 4AR; 44% average ↑ over Equol] |

Note:
4AR and Equol results from prior clinical study data (Naftolin & Lephart, 2021), whereas, CDB + Polyphenol (Equol) are from the 1^{st} Skin Study and Polyphenol (Eauol) Plus Natural Ingredients are from clinical study II data. When Equol plus the natural ingredient results were compared to 4 AR results (alone) on average there was a significant 30% increase in efficacy and when compared to the Equol results (alone) on average there was a significant 44% increase in efficacy.
4AR (alone) Study details: Randomized, single center 12-week study; N = 36 (24 Caucasian, 12 Asian); Ages 34-64; Mean age 53.5 ± 6.2 (20 out of 36 or 56% were amenorriheic for at least 2 years); Glogua Aging II and III (mild to moderate wrinkling); Applied twice daily
Equol (alone) Study details: Randomized, single center 12-week study; N = 59 (30 Caucasian, 29 Asian); Ages 40-70; Mean age 56.1 ± 7.8 (45 out of 59 or 76% were amenorriheic for at least 3 years); Glogua Aging II and III (mild to moderate wrinkling) Applied twice daily
In comparing the clinical parameters of the 4AR to the equol technology results showed that: a) in general, the percent improvement in the 8 skin areas for both treatment were similar, b) the slightly higher percentages for some of the skin parameters for the 4AR vs. the equol technology maybe due to the difference in the number of postmenopausal women, where the equol study had 20% more female subjects that were amenorriheic for at least 3 years compared to the 4AR subjects and, c) the concentration of the 4AR treatment was more than 3-times that of the equol treatment at: 1.0% vs. 0.3%, respectively.

As shown in Table 22 for these formulation comparisons the CDB plus Equol (e.g. SF1 or SF2) of the Equol plus the natural ingredient formulations (STF6 and STF7) demonstrated substantially significant improvement in the efficacy of all the quantified skin parameters for these ingredient combinations over the individual ingredients alone, such as 4′-acetoxy resveratrol (4AR) or equol.

Notably, in the second clinical skin study there were 8% more women that were post-menopausal compared to the first clinical skin study, while the number of subjects in both clinical studies was approximately the same. This demonstrated the strength of the combination treatments that included CDB and Equol along with the natural ingredient combinations, such as, vitamin C, grape seed extract and hyaluronic acid mixtures (Table 22).

These findings are not only important for the applications to men's and women's skin health but, especially for women who experience perimenopausal and postmenopausal intervals when dermal health dramatically declines with aging (Farage et al., 2008; Farage, Miller & Maibach, 2020; Lephart, 2018b; Reus et al., 2020; Stevenson & Thornton, 2007). In fact, a review (reported in the Journal of Cosmetic Dermatology) demonstrated that the decline in estrogen levels during perimenopause and the post-menopausal intervals are directly linked to an increase in dermal aging and a dramatical decline in facial attractiveness (Lephart, 2018b).

The findings described herein surprisingly show that the combination of CBD and/or polyphenols (such as equol) along with other natural active ingredients result in a significantly greater (via statistical analysis) and surprising results compared to when CBD, polyphenols (i.e., equol, resveratrol, etc.) or the other natural active ingredients were used alone as treatments for various disorders, diseases, and biological functions (Table 22).

Although several aspects of the invention have been discussed in detail above, the scope of the invention is not limited to what has been presented herein, and instead, is determined by the following claims.

What is claimed:

1. A composition comprising:
cannabidiol (CBD); and
at least one of:
one or more polyphenolic compound, its isomer or analog; and
one or more other active ingredient selected from the group consisting of collagen, vitamin C, vitamin E, zinc, grape seed extract, niacinamide, hydroxy acids, hyaluronic acid, and coenzyme Q10.

2. A composition comprising:
at least one polyphenolic compound, its isomer or analog; and
at least one other active ingredient selected from the group consisting of collagen, vitamin C, vitamin E, Zinc, grape seed extract, niacinamide, hydroxy acids, hyaluronic acid, and coenzyme Q10.

3. The composition of claim 2, wherein the composition is in a form of lotion, spray, foam, gel, or trans-dermal patch, and is administered topically.

4. The composition of claim 2, wherein the composition is in the form of a liquid, tablet, capsule, dietary or nutritional supplement, and is administered orally.

5. The composition of claim 2, wherein the composition is administered by an intra-muscular injection or intra-venous methods.

6. The composition of claim 2, wherein the composition is administered as a food, supplement or OTC product, medicinal food, or medicinal supplement.

7. The composition of claim 2, wherein the at least one phytochemical compound is selected from the group consisting of racemic and non-racemic equol, resveratrol, and astaxanthin,
wherein, the racemic or non-racemic equol forms about 0.1% to 10% of the composition for topical uses, and from about 1 mg to 15 mg for oral uses;
wherein resveratrol forms from about 0.1% to 10% of the composition for topical uses, and from about 1 mg to 500 mg for oral uses; and
wherein astaxanthin forms about 0.1% to 10% of the composition for topical uses, and from about 1 mg to about 12 mg for oral uses.

8. The composition of claim 2, wherein the at least one other active ingredient forms from about 0.1% to 25% of the composition for topical uses, and from about 1 mg to 2500 mg for oral uses.

9. A method for the prevention, treatment and/or ameliorating of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject, comprising:
administering to the subject a composition comprising:
one or more polyphenolic compound, its isomer or analog; and
one or more other active ingredient selected from the group consisting of collagen, vitamin C, vitamin E, zinc, grape seed extract, niacinamide, hydroxy acids, hyaluronic acid, and coenzyme Q10.

10. The method of claim 9, wherein:
the at least one condition or disorder of skin and accessory structures of the skin is skin irritation (dermatitis), psoriasis, eczema, skin cancer (melanoma), skin aging-chronological (intrinsic) and photo (extrinsic) aging, augmentation of collagen, elastin, wound healing, facial color and tone, skin smoothness and integrity (barrier repair) including finger nail growth and repair, defense against matrix metalloproteinases, elastase, fibroblast collapse, inflammatory biomarkers, acne, scalp hair loss, thinning of the skin and scalp hair retention;
the at least one condition or disorder of musculoskeletal system is muscle and joint pain, muscle cramps, movement disorder, osteoporosis, and neuromuscular condition;
the at least one condition or disorder of the cardiovascular system is hypertension, heart disease, stroke, tissue hypoxia and blood vessel integrity;
the at least one condition or disorder of the inflammatory system is defending against oxidative stress, oxygen free radicals, inhibition of NFKappB, protecting against oxidative and enhancement of antioxidant protection;
the at least one condition or disorder of the neurological system is memory loss, decreased cognitive function, neurodegenerative disorders (Alzheimer's and Parkinson's disease), white matter brain lesions with aging or trauma, cranial blood vessel integrity and fibromyalgia;
the cancer is skin cancer, DNA mutation, radiation burns, radiation sickness, and premature aging and other tissue and organ cancers; and
the at least one condition or disorder of the endocrine/metabolic system is weight control, hypercholesterolemia, osteoporosis, pituitary disorder, adrenal disorder, pancreatic disorder (diabetes, insulin) and thyroid disorder.

11. The method of claim 9, wherein the composition is in a form of lotion, spray, foam, gel, or trans-dermal patch and is administered topically.

12. The method of claim 9, wherein the composition is in the form of a liquid, tablet, capsule, dietary or nutritional supplement, and is administered orally.

13. The method of claim 9, wherein the composition is administered by an intra-muscular injection or intra-venous methods.

14. The method of claim 9, wherein the composition is administered as a food, supplement or OTC product, medicinal food, or medicinal supplement.

15. The method of claim 9, wherein the composition is administered to the subject at a dose of about 0.1% to about 20% by weigh of the composition.

16. The method of claim 9, wherein the composition is administered to the subject at a dose of about 0.1 to about 1,500 mg.

17. The method of claim 9, wherein the composition is administered to the subject at a dose of about 0.1 to about 500 mg.

18. The method of claim 9, wherein the composition is administered to the subject at a dose of about 0.1 to about 100 mg.

19. The method of claim 9, wherein the composition is administered to the subject at a dose of about 10 to about 1,500 mg.

20. The method of claim 9, wherein the composition is administered to the subject at a dose of about 10 to about 2,500 mg.

21. The method of claim 9, wherein the at least one phytochemical compound is selected from the group consisting of racemic equol or non-racemic equol, resveratrol, and astaxanthin.

22. A method for the prevention and/or treatment of at least one condition or disorder of a) skin and accessory structures of the skin, b) musculoskeletal system, c) cardiovascular system, d) inflammatory system, e) neurological system, f) cancer, and g) endocrine/metabolic system, and/or for improving the health status of a subject, comprising:
administering to the subject a composition comprising:
cannabidiol (CBD); and
at least one of:
one or more polyphenolic compound, its isomer or analog; and
one or more other active ingredient selected from the group consisting of collagen, vitamin C, vitamin E, zinc, grape seed extract, niacinamide, hydroxy acids, hyaluronic acid, and coenzyme Q10,
wherein the CBD binds the CBD receptors and the at least one polyphenolic compound binds the polyphenolic receptors.
